(19) United States
(12) Patent Application Publication
(10) Pub. No.: US 2022/0280473 A1
(43) Pub. Date: Sep. 8, 2022

(54) COMPOSITIONS AND METHODS FOR MODULATING EPITHELIAL-MESENCHYMAL TRANSITION

(71) Applicants: Brian LEVY, Tampa, FL (US); AUCKLAND UNISERVICES LIMITED, Auckland (NZ)

(72) Inventors: Colin Richard GREEN, Auckland (NZ); Odunayo Omolola Boluwarin MUGISHO, Auckland (NZ); Brian LEVY, Tampa, FL (US)

(21) Appl. No.: 17/685,268

(22) Filed: Mar. 2, 2022

(30) Foreign Application Priority Data
Mar. 2, 2021 (NZ) 773493

(51) Int. Cl. A61K 31/352

(52) U.S. Cl. CPC A61K 31/352

(57) ABSTRACT

The inventions relate to the use of anti-hemichannel compounds, including anti-connexin 43 hemichannel opening compounds, inhibitors and blockers, to modulate, suppress and stabilize epithelial-mesenchymal and/or endothelial-mesenchymal transition in diseases, disorders and conditions, including fibrotic diseases, disorders and conditions and other conditions associated with fibrosis.

### FIELD

The inventions relate generally to connexin hemichannels, and to compositions and methods to inhibit epithelial-mesenchymal transition in disease or otherwise pathological or abnormal levels of epithelial-mesenchymal transition. The inventions relate to the use of anti-hemichannel compounds, including anti-connexin 43 hemichannel opening compounds, inhibitors and blockers, to modulate, inhibit, suppress and stabilize pathological or otherwise unwanted epithelial-mesenchymal transition.

### INCORPORATION BY REFERENCE

All U.S. patents, U.S. patent application publications, foreign patents, foreign and PCT published applications, articles and other documents, references and publications noted herein, and all those listed as References Cited in any patent or patents that issue herefrom, are hereby incorporated by reference in their entirety. The information incorporated is as much a part of this application as if all the text and other content was repeated in the application and will be treated as part of the text and content of this application as filed.

### BRIEF BACKGROUND

The following includes information that may be useful in understanding the present inventions. It is not an admission that any of the information, publications or documents specifically or implicitly referenced herein is prior art, or essential, to the presently described or claimed inventions.

The ability of epithelial cells and endothelial cells to transform into mesenchymal cells is a well-known cellular mechanism. This process, referred to as epithelial-mesenchymal transition (EMT) or endothelial-mesenchymal transition (EndMT), regulates various stages of embryonic development, but also contributes to the progression of a wide array of diseases. See J. P. Thiery, et al., “Epithelial-mesenchymal transitions in development and disease,” Cell vol. 139, no. 5, pp. 871-890, 2009; R. Kalluri and R. A. Weinberg, “The basics of epithelial-mesenchymal transition,” J. Clin. Invest., vol. 119, no. 6, pp. 1420-1428, 2009. In EMT polarized epithelial cells acquire motile mesothelial phenotypic features. It is a multi-step process whereby polarized epithelial cells change phenotype until they become mesenchymal (Kalluri & Weinberg, 2009). These changes range from the activation and deactivation of transcription factors and expression of specific mRNAs, to changes in the expression and structure of cytoskeletal and cell-surface proteins (Kalluri & Weinberg, 2009). Transitioning cells demonstrate both epithelial and mesenchymal phenotypes, with their respective proportions shifting as the process progresses. Despite a common progression, the conditions under which EMT occurs have been split into three types. “Type 1” EMT occurs during implantation, embryogenesis and organ development, while “type 3” EMT occurs in neoplastic cells and is linked to cancer progression and metastasis. “Type 2” is associated with organ fibrosis, tissue regeneration and wound healing and occurs frequently in tissues following trauma and/or inflammation.

During embryogenesis, EMT is essential for gastrulation, primitive streak formation, somite dissociation, neural crest development, and palate and lip fusion. EndMT is critical for cardiac development, particularly in the formation of the valves and septa of the heart and the generation of mesodermal cells and multipotent progenitors.

In the adult organism, EMT and EndMT are usually dormant until pathological stimuli awaken this embryonic mechanism. For example, EMT is the primary mechanism of cancer metastasis (G. P. Gupta and J. Massagué, “Cancer metastasis: building a framework,” Cell, vol. 127, no. 4, pp. 679-695, 2006; J.-Y. Shih and P.-C. Yang, “The EMT regulator slug and lung carcinogenesis,” Carcinogenesis, vol. 32, no. 9, pp. 1299-1304, 2011), whereas EndMT forms cancer-associated fibroblasts in the tumor microenvironment. S. Potenta, et al., “The role of endothelial-to-mesenchymal transition in cancer progression,” British Journal of Cancer, vol. 99, no. 9, pp. 1375-1379, 2008. Also, both EMT and EndMT have been shown to generate fibroblasts that cause the formation of scar tissue after tissue injury or in association with inflammatory and fibrotic diseases. R. Kalluri and E. G. Neilson, “Epithelial-mesenchymal transition and its implications for fibrosis,” J. Clin. Invest., vol. 112, no. 12, pp. 1776-1784, 2003; S. Piera-Velazquez, et al., “Role of endothelial-mesenchymal transition (EndoMT) in the pathogenesis of fibrotic disorders,” American Journal of Pathology, vol. 179, no. 3, pp. 1074-1080, 2011; P. Pessina, et al., “Fibrogenic cell plasticity blunts tissue regeneration and aggravates muscular dystrophy,” Stem Cell Reports, vol. 4, no. 6, pp. 1046-1060, 2015. Mesenchymal transitions have traditionally been considered to have a positive effect in development and a negative effect in disease. Many studies have proposed that induction of EMT is the primary mechanism by which epithelial cancer cells acquire malignant phenotypes that promote metastasis.

EMT has also been observed in retinal pigment epithelial cells following insult (Lee et al., 2020; Yang et al., 2020; Che et al., 2016; Chen et al., 2014). Epithelial-mesenchymal transition in retinal pigment epithelial cells is also related to the pathogenesis of subretinal fibrosis such as that associated with macular degeneration. RPE cells form the outer blood-retinal barrier (BRB) at the back of the eye and have high functional importance, regulating retinal glucose homeostasis, photoreceptor functionality and angiogenic balance, and disruption to RPE function has been implicated in multiple ocular diseases including diabetic retinopathy (DR), age-related macular degeneration (AMD) and proliferative vitreoretinopathy (PVR) (Hyttinen et al., 2019; Chen et al., 2014; Che et al., 2016). Of these, PVR has already been linked with EMT (Tamiya & Kaplan, 2016), while high glucose, characteristic in diabetic conditions, has also been found to induce EMT in RPE cells (Che et al., 2016). The mechanism by which EMT is induced in RPE cells is still debated, although multiple disease pathways have been linked to its activation. Both high glucose (Che et al., 2016) and TGF-β₂ (Mony et al., 2013; Chen et al., 2014) have independently been shown to induce EMT in RPE cells. In particular, high glucose was found to increase expression of one of the key EMT transcription factors, Snail (Che et al., 2016).

Proliferative vitreoretinopathy is a severe blinding complication of rhegmatogenous retinal detachment. Epithelial-mesenchymal transition of RPE cells is thought to play a pivotal role in the pathogenesis of PVR. Epithelial-mesenchymal transition (EMT), which enables RPE cells to lose their epithelial properties and transform into mesenchymal cells, is considered as the fundamental mechanism underlying the formation of the PVR membrane. Similar to EMT in carcinogenesis, the EMT of RPE cells involves the activation of the relevant cellular pathway, rearrangement of the cytoskeleton, and disassembly of the junctions between RPE cells. Transforming growth factor (TGF)-β, a classic EMT trigger, is also found in the eye of PVR patients. Therefore, blocking the EMT of RPE cells will be an efficient way to prevent PVR. However, despite the study of the EMT of RPE cells in PVR for decades, there is no currently available drug to prevent it.

EMT has also been observed in the corneal endothelium of the eye, with primary changes in acquired or inherited corneal disease including loss of endothelial cell density and change in morphology to a fibroblastic cell type. Inherited disease includes Fuch's endothelial corneal dystrophy, the most common corneal endothelial dystrophy and leading to loss of vision. Acquired disease includes pseudophakic or Aphakic bulbous keratoplasty, and failed previous corneal grafts.

Descemet stripping endothelial keratoplasty (DSEK) or automated DSEK (DAESK) are procedures of choice in many centers for corneal endothelial repair which is essential to retain corneal clarity and viability, but is constrained by limited donor tissue availability. Tissue engineering is being used to build artificial conical tissue but EndoMT of endothelial cells remains a challenge.

We studied the potential role of connexins and connexin hemichannels in EMT, including connexin 43 and connexin 43 hemichannels. We discovered connexin hemichannel modulation agents are anti-EMT agents that can be used to modulate EMT, and serve as a therapeutic for EMT, as well as EndMTin disease, including in retinal diseases that include those characterized by subretinal fibrosis and others. This patent relates to the important discovery of methods and compositions comprising anti-hemichannel compounds that can modify and inhibit epithelial-mesenchymal transition in EMT-related diseases, disorders and conditions.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this introduction, which is included for purposes of illustration only and not restriction.

This patent is directed to methods and compositions and the use of anti-hemichannel compounds to inhibit epithelial-mesenchymal transition (EMT). The patent is also directed to methods and compositions and the use of anti-hemichannel compounds to maintain proper (non-pathological) levels of EMT. The patent is further directed to methods and compositions and the use of anti-hemichannel compounds to inhibit endothelial-mesenchymal transition (EndMT). The patent is also directed to methods and compositions and the use of anti-hemichannel compounds to maintain proper (non-pathological) levels of EndMT.

Data herein show, for example, that anti-hemichannel compounds can be used to inhibit EMT, including in chronic retinal diseases, conditions and disorders. It was also discovered that anti-hemichannel compounds can be used to maintain EMT at non-pathological levels. Anti-hemichannel compounds can also be used to maintain endothelial-mesenchymal transition (EndMT) at non-pathological levels.

Thus, in one aspect, the invention provides for the use of anti-hemichannel compounds to inhibit epithelial-mesenchymal transition (EMT). In another aspect, the invention provides for the use of anti-hemichannel compounds to inhibit endothelial-mesenchymal transition (EndMT).

In another aspect, the invention provides methods for regulating EMT in the retina.

In another aspect, the invention provides methods for regulating EMT in the retina pigment epithelium.

In another aspect, the invention provides methods for regulating EMT in retina pigment epithelium cells.

In another aspect the invention provides methods for regulating EndMT in the cornea.

In another aspect the invention provides methods for regulating EndMT in corneal endothelial cells.

In another aspect, the provides methods for regulating EMT in cancer.

In another aspect, the provides methods for regulating EMT in fibrotic diseases, disorders and conditions.

The patent also describes the use of orally-delivered anti-hemichannel compounds for inhibiting EMT in afflicted patients, the use of orally-delivered anti-hemichannel compounds for and reversing or substantially reversing EMT-related disease.

The patent also describes the use of orally-delivered anti-hemichannel compounds for rescuing normal EMT function in patients in need suffering from chronic ocular disease.

The patent also describes the use of orally-delivered anti-hemichannel compounds for inhibiting EMT in patients in need suffering from chronic ocular disease characterized at least in part by dysregulated EMT.

The patent is also directed to methods for the use of anti-hemichannel compounds for these purposes, including, for example, tonabersat, a benzopyran compound (cis-6-acetyl-4S-(3-chloro-4-fluoro-benzoylamino)-3,4-dihydro-2,2-dimethyl-2H-benzo[b]pyrane-3 S-ol (SB-220453, also referred to as Xiflam or tonabersat), as well as tonabersat pro-drugs (see, e.g., the compounds of Formula II).

The inventions relate, in one aspect, for example, to the use of anti-hemichannel compounds to treat EMT dysregulation in a subject with conditions characterized in whole or in part by pathological or otherwise unwanted EMT activity, including diabetic retinopathy, age-related macular degeneration and proliferative vitreoretinopathy.

In some methods the EMT modulation or inhibition treats a chronic retinal disorder. In other aspects, the chronic retinal disorder is diabetic retinopathy, age related macular degeneration or proliferative vitreoretinopathy. In other aspects the increasing survival methods treat a retinal or other disorder characterized by a pathological or otherwise unwanted level of EMT activity.

In some methods the EMT modulation or inhibition (or EndMT modulation or inhibition) treats a fibrosis/fibrotic disorder. In one embodiment, the EMT modulation or inhibition treats an ocular fibrosis disorder. Epithelial-mesenchymal transition has become widely accepted as a mechanism by which injured renal tubular cells transform into mesenchymal cells that contribute to the development of fibrosis in the kidney and in chronic renal failure, and in some embodiments the EMT modulation or inhibition (or EndMT modulation or inhibition) using, for example, compounds and methods to modulate connexin hemichannels, including connexin 43 hemichannels, treats kidney fibrosis. In some embodiments, the EMT modulation or inhibition (or EndMT modulation or inhibition) treats renal failure or chronic renal failure. In some embodiments, the EMT modulation or inhibition (or EndMT modulation or inhibition) treats EMT and/or EndMT in renal epithelial cells following kidney injury. In another embodiment of the method, the EMT- or EndMT-related disease, disorder or condition in the subject is a cancer. In some embodiments the EMT modulation or inhibition (or EndMT modulation or inhibition) using, for example, compounds and methods to modulate connexin hemichannels, including connexin 43 hemichannels, treats kidney fibrosis. In some embodiments, the EMT modulation or inhibition (or EndMT modulation or inhibition) treats renal failure or chronic renal failure. In some embodiments, the EMT modulation or inhibition (or EndMT modulation or inhibition) treats EMT in renal epithelial cells following kidney injury. In some embodiments the EMT modulation or inhibition (or EndMT modulation or inhibition) treats fibrosis in organs other than the eye and kidney. In some embodiments, the EMT modulation or inhibition (or EndMT modulation or inhibition) treats fibrosis following inflammation. In some embodiments, the EMT modulation or inhibition (or EndMT modulation or inhibition) treats EMT or EndMT in renal epithelial or endothelial cells following kidney injury. In some embodiments, the EMT modulation or inhibition (or EndMT modulation or inhibition) treats any fibrotic disorder. As used herein, fibrotic disorders include and any disease, disorder or condition where epithelial cells are induced to acquire a myofibroblast phenotype and ultimately a fibrotic phenotype. EMT- and EndMT-related fibrotic disorders treatable with compounds and methods of the invention include, for example, pulmonary (lung) fibrosis, kidney fibrosis, idiopathic pulmonary fibrosis, liver fibrosis (including hepatic fibrosis resulting from hepatitis B and C, nonalcoholic steatohepatitis, and alcohol abuse), intestinal fibrosis, ocular fibrosis, adipose tissue fibrosis, cardiac and other organ fibroses, as well as scleroderma.

This patent describes, in one aspect, the use of compounds and methods to modulate connexin hemichannels, including connexin 43 hemichannels, to inhibit EMT activity. It also describes the use of compounds and methods to modulate connexin hemichannels, including connexin 43 hemichannels, to maintain normal EMT activity.

This patent describes, in one aspect, the use of compounds and methods to modulate connexin hemichannels, including connexin 43 hemichannels, to inhibit EMT caused by acute or chronic systemic hyperglycemia.

Anti-hemichannel compounds useful in the present invention include compounds of Formula I, for example Xiflam (tonabersat), and/or a prodrug of any of the foregoing compounds, and other anti-hemichannel compounds described or incorporated by reference herein. In some embodiments, the hemichannel blocker is a small molecule other than Xiflam (tonabersat), for example, a hemichannel blocker described in Formula I or Formula II in US Pat. App. Publication No. 20160177298, filed in the name of Colin Green, et al., the disclosure of which is hereby incorporated in its entirety by this reference.

In one embodiment, the compound used to modulate connexin hemichannels is a compound according to Formula I.

In another embodiment, the compound used to modulate connexin hemichannels is a compound according to Formula II.

In one embodiment, the compound used to modulate connexin hemichannels is a peptide hemichannel inhibitor. In one embodiment, the compound used to modulate connexin hemichannels is a connexin 43 peptidomimetic. Useful connexin 43 peptidomimetics include, for example, Peptide 5, GAPS, GAP19, GAP26, GAP27 or α-connexin carboxy-terminal (ACT) peptides, e.g., ACT-1 or other active anti-hemichannel peptidomimetics.

In one embodiment, the compound used to modulate connexin hemichannels is a peptide construct comprising (a) a targeting carrier peptide derived from the X-protein of the Hepatitis B virus and (b) a peptide capable of interacting with an intracellular domain of connexin43 (Cx43), for example, XG19, as described in PCT Application No. PCT/NZ2018/050059 (“Methods of Treatment and Novel Constructs”), the disclosure of which is hereby incorporated in its entirety by this reference.

It is another object of the invention to provide compounds, compositions, formulations, kits, doses and methods for the treatment of diseases, disorders and conditions that will benefit from inhibition of EMT, and/or restoration of normal EMT activity.

In some aspects, the method of treatment is applied to mammals, e.g., humans.

Although hemichannel inhibitors may be delivered using any art-known method, some preferred embodiments include use of an orally available small molecule anti-hemichannel compound, to inhibit EMT activity in subjects who are or may be at risk for loss of retinal and/or choroidal structure or function.

Other aspects of the invention include methods of inhibiting or modulating EMT in a subject having a chronic retinal disorder, comprising administering an effective amount of a hemichannel blocker to said subject.

Included are methods for inhibiting or modulating EMT in a subject in need thereof, comprising, e.g., administering to said subject an EMT inhibiting amount of N-[(3S,4S)-6-acetyl-3-hydroxy-2,2-dimethyl-3,4-dihydrochromen-4-yl]-3-chloro-4-fluorobenzamide (Xiflam). In some embodiments, the inhibiting amount is about 50 to about 250 mg per dose or per day. In other embodiments, the survival-promoting amount is about 80 to about 320 mg, 400 mg, 500 mg or up to about 1000 mg per day. These amounts may be administered in single or divided doses, e.g., BID. Other daily doses, as well as particularly useful weekly, monthly and implant dosing and dosing regimens are provided herein. In various embodiments, the small molecule that blocks or ameliorates or inhibits hemichannel opening is a prodrug of Xiflam (tonabersat) or an analog thereof.

In another aspect, the invention provides the use of a hemichannel blocker in the manufacture of a medicament for use in the treatment of subjects, or of the diseases, disorders and conditions, described or referred to herein. The medicament will comprise, consist essentially of, or consist of an anti-hemichannel compound. In one embodiment, the anti-hemichannel compound is a small molecule anti-hemichannel compound. In another embodiment, the small molecule anti-hemichannel compound an orally-available small molecule anti-hemichannel compound.

In other aspects of methods of the invention, EMT is improved or normalized.

In another aspect, the invention provides the use of a hemichannel blocker in the manufacture of a medicament (or a package or kit containing one or more medicaments and/or containers, with or without instructions for use) for modulation of a hemichannel and treatment of an EMT-related disease, disorder and/or condition, including any of the diseases, disorders and/or conditions described or referred to herein. In one aspect, for example, the invention provides the use of a small molecule connexin hemichannel blocker, including, for example, Xiflam and/or an analogue or prodrug thereof. In one embodiment, the medicament will comprise, consist essentially of, or consist of a connexin 43 hemichannel blocker, for example, a small molecule connexin 43 hemichannel blocker. In one embodiment, the hemichannel blocker composition useful in the invention may include a pharmaceutically acceptable carrier and may be formulated as a pill, a solution, a microsphere, a liposome, a nanoparticle, an implant (including, for example, peritoneal, subcutaneous and ocular implants, as well as slow- or controlled-release implants), a matrix, or a hydrogel formulation, for example, or may be provided in lyophilized form.

The hemichannel being modulated for the purposes described herein may be any connexin of interest for that purpose. For example, the hemichannel being modulated for the purposes described herein may be a connexin hemichannel expressed in the retina, in blood vessels, and/or in the vascular wall. In one embodiment the hemichannel blocker blocks a connexin hemichannel in a blood vessel. In other embodiments the hemichannel blocker blocks a connexin hemichannel in a blood microvessel. In other embodiments the hemichannel blocker blocks a connexin hemichannel in a capillary.

In other embodiments the hemichannel blocker blocks a connexin hemichannel in the epithelium or in the endothelium.

In various embodiments, by way of example, the hemichannel being modulated comprises one or more of connexin 36 (Cx36), connexin 37 (Cx37), connexin 40 (Cx40), connexin 43 (Cx43), connexin 45 (Cx45), connexin 57 (Cx57), connexin 59 (Cx59) and/or connexin 62 (Cx62).

In one embodiment, particularly as it relates to the retina, the hemichannel being modulated comprises one or more of a Cx36, Cx37, Cx40, Cx43, Cx45 or Cx57 protein. Targeted hemichannel connexins include one or more of selected hemichannel connexins in blood vessels (e.g., Cx37, Cx40 or Cx43), as well as hemichannel connexins in astroglial cells (e.g., Cx43), amacrine cells (e.g., Cx36, Cx45), bipolar cells (e.g., Cx36, Cx45), the outer and inner plexiform layer, the ganglion cell layer (e.g., Cx36, Cx45), cone photoreceptors and retinal endothelial cells, and other retinal neurons, for example. In some embodiments, Cx36 and Cx43 hemichannels are targeted. In one particular embodiment, the hemichannel and/or hemichannel being modulated comprises Cx43. In one embodiment, hemichannels comprising connexins in the cells of the outer plexiform layer are targeted (e.g., Cx43).

In other embodiments, particularly those relating to the choroid or blood vessels of the retina, the hemichannel being modulated may preferentially comprise one or more of a Cx37, Cx40 or Cx43 protein. In one particular embodiment, the hemichannel and/or hemichannel being modulated comprises Cx43. In one embodiment, hemichannels comprising vessel connexins in cells of the outer choroid, also known as Haller's layer, which is composed of large caliber, non-fenestrated vessels, are targeted. In another embodiment, hemichannels comprising vessel and endothelial cell connexins in cells of the inner choroid, also known as Sattler's layer, which is composed of significantly smaller vessels, are targeted. In another embodiment, hemichannels comprising connexins in cells of the outer and inner choroid are targeted. In another embodiment, hemichannels comprising connexins in capillaries of the choriocapillaris are targeted. In one embodiment, hemichannel vessel connexins targeted in methods of the invention include hemichannel connexins in pericytes and connexins in vascular smooth muscle and endothelial cells. In another embodiment, hemichannel vessel connexins targeted in methods of the invention include hemichannels in pericytes and connexins in endothelial cells, for example, in the microcapillaries. Cx43 hemichannels are a preferred target of the invention.

One method of the invention comprises the steps of (1) identifying a subject with an EMT-related disease, disorder or condition, (2) administering a therapeutically effect amount of a connexin hemichannel inhibitor to the subject and, optionally, (3) measuring or visualizing EMT activity the subject. In one embodiment, the EMT activity is measured or visualized and the dose is maintained or adjusted. In one embodiment of the method, step (1) is not required because the subject is already known to have an EMT-related disease. In one embodiment, the disease, disorder or condition is an EndMT-related disease, disorder or condition. In one embodiment, EMT and/or EndMT is lessened, inhibited or otherwise attenuated. In one embodiment of the method, the connexin hemichannel inhibitor is a connexin 43 hemichannel inhibitor. In one embodiment of the method, the connexin 43 hemichannel inhibitor is a small molecule connexin 43 hemichannel inhibitor. In another embodiment of the method, the connexin hemichannel inhibitor is an anti-connexin 43 hemichannel peptide or peptidomimetic that inhibits or blocks connexin 43 hemichannel opening or activity. In one embodiment of the method, the connexin 43 hemichannel inhibitor is tonabersat. In another embodiment of the method, the connexin 43 hemichannel inhibitor is carabersat. In one embodiment of the method, the EMT-related disease, disorder or condition in the subject is characterized by EMT dysregulation. In one embodiment of the method, the EMT-related disease, disorder or condition in the subject is characterized in whole or in part by pathological or otherwise unwanted EMT activity. In one embodiment of the method, the EMT-related disease, disorder or condition in the subject is diabetic retinopathy, age-related macular degeneration or proliferative vitreoretinopathy. In another embodiment of the method, the EMT-related disease, disorder or condition in the subject is a retinal or other disorder characterized by a pathological or otherwise unwanted level of EMT activity. In another embodiment of the method, the EMT-related disease, disorder or condition in the subject is Fuchs endothelial corneal dystrophy, or Pseudophakic or Aphakic keratopathy. In another embodiment of the method, the EMT-related disease, disorder or condition in the subject is a fibrosis disorder. In one embodiment, the EMT modulation or inhibition treats an ocular fibrosis disorder. In another embodiment of the method, the EMT-related disease, disorder or condition in the subject is a cancer or a renal disease or injury.

Another embodiment of this aspect of the invention provides a pharmaceutical pack that includes a small molecule or other hemichannel blocker. In one embodiment, the hemichannel blocker is Xiflam (tonabersat). In another embodiment, the hemichannel blocker in the pharmaceutical pack comprises, consists essentially of, or consists of Peptide5, GAP9, GAP19, GAP26, GAP27 or α-connexin carboxy-terminal (ACT) peptides, e.g., ACT-1 or other active anti-hemichannel peptidomimetics, for example.

The activity of hemichannel blockers may be evaluated using certain biological assays. Effects of known or candidate hemichannel blockers on molecular motility can be identified, evaluated, or screened for using the methods described in the Examples below that use human adult retinal pigment epithelial cells, or other art-known or equivalent methods for determining the passage of compounds through connexin hemichannels. Various methods are known in the art, including dye transfer experiments, for example, transfer of molecules labelled with a detectable marker, as well as the transmembrane passage of small fluorescent permeability tracers, which has been widely used to study the functional state of hemichannels. Various embodiments of this aspect of the invention are described herein, including a method for use in identifying or evaluating the ability of a compound to block hemichannels, which comprises: (a) bringing together a test sample and a test system, said test sample comprising one or more test compounds, and said test system comprising a system for evaluating hemichannel block, said system being characterized in that it exhibits, for example, elevated transfer of a dye or labelled metabolite, for example, in response to the introduction of hypoxia or ischemia to said system, a mediator of inflammation, or other compound or event that induces hemichannel opening, such as a drop in extracellular Ca²⁺; and, (b) determining the presence or amount of a rise in, for example, the dye or other labelled metabolite(s) in said system. Positive and/or negative controls may be used as well. Optionally, a predetermined amount of hemichannel blocker (e.g., Xiflam) may be added to the test system. Other methods useful to evaluate hemichannel blocker activity include electrophysiology and channel conductance block techniques, reduction in cytoplasmic swelling or cell edema, and reduced potassium efflux from cells, all of which are known in the art.

In one aspect, methods are provided for confirming, measuring or evaluating the activity of compounds useful for restoring or rescuing retinal function using assays, including tests using ARPE-19 cells. See Dunn K C, et al., ARPE-19, a human retinal pigment epithelial cell line with differentiated properties. Exp Eye Res. 1996 February; 62(2):155-69. Art methods may be used for confirming, measuring or evaluating the activity of compounds useful for inhibiting EMT and EndMT activity, including ultrasonography, magnetic resonance imaging (MRI), and enhanced depth imaging optical coherence tomography (EDI-OCT) and swept-source OCT (SS-OCT).

In one aspect, methods are provided for confirming, measuring or evaluating the activity of compounds useful for restoring or rescuing corneal endothelium function using assays, including tests using B4G12 cells. Art methods, including in vivo confocal microscopy, corneal pachymetry, contact and non-contact specular photo microscopy (see Gasser, L., Reinhard, T. & Böhringer, D. Comparison of corneal endothelial cell measurements by two non-contact specular microscopes. BMC Ophthalmol 2015; 15:87) may be used for confirming, measuring or evaluating the activity of compounds useful for restoring or rescuing corneal endothelial function.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 HG+Cyt treatment induced a change in phenotype in ARPE-19 cells over 72 h, with an increasing effect over time. Untreated cells have a truncated fibroblastic or cuboidal form. HG+Cyt treated cells become elongated and stretched. Scale bar=50 μm.

FIG. 2 (a, b) RPE65 expression was decreased following HG+Cyt insult, but prevented by co-application of a hemichannel inhibitor of Formula I, tonabersat (HG+Cyt+Ton). (c, d) α-SMA expression was increased and appeared more striated following HG+Cyt insult compared to untreated conditions, but changes were prevented by addition of tonabersat (HG+Cyt+Ton). n=4; boxed areas are enlarged; scale bars=50 or 25 μm.

FIG. 3 (a) Quantification of RPE65 expression by mean fluorescent intensity showed that RPE65 expression was significantly higher in the HG+Cyt+Ton group compared to the HG+Cyt group at 24 h (p=0.0286). (b) By 72h, HG+Cyt had significantly reduced RPE65 expression relative to untreated conditions (p=0.0040), however, there was no statistically significant difference between HG+Cyt+Ton and HG+Cyt groups. (c) At 24 h, α-SMA expression was significantly higher following HG+Cyt than untreated conditions (p=0.0459). (d) By 72 h, the HG+Cyt+Ton treatment group had significantly reduced α-SMA expression levels compared with HG+Cyt insult alone (p=0.0119). (e) The proportion of α-SMA to RPE65 expression was noticeably higher following HG+Cyt insult in comparison to untreated and HG+Cyt+Ton conditions, after both 24 and 72 h of incubation. At 24 h, the untreated group had an α-SMA to RPE65 ratio of 1.0, HG+Cyt a ratio of 1.7, and HG+Cyt+Ton a ratio of 1.1. Similar trends were seen at 72 h with the untreated and HG+Cyt+Ton groups having similar ratios of 1.0 and 0.8, while the HG+Cyt insult group was up at 1.5. Dashed line indicates a ratio of 1.0. Statistical analysis was carried out using one-way ANOVA with Dunnett's multiple comparison test. *p≤0.05; **p≤0.01; n=4. In FIGS. 3, 6, and 7, the basal condition is indicated in black (left bar), the HG+Cyt condition is represented in red (middle bar), and the HG+Cyt+Ton condition is represented in green (right bar).

FIG. 4 (a) HG+Cyt+Ton treatment prevents HG+Cyt-induced tight junction loss. ZO-1 localization was seen to be influenced by the treatment conditions, with a loss of localization to the cell membrane seen following HG+Cyt insult. Addition of tonabersat (HG+Cyt+Ton) prevented loss of ZO-1 cell membrane localization, although a small amount of internalization still occurred relative to the untreated group. (b) HG+Cyt+Ton treatment prevents HG+Cyt-induced loss of Cx43 localization, which was reversed by addition of exogenous ATP. Cx43 cell membrane localization was reduced by HG+Cyt conditions, while addition of tonabersat maintained Cx43 localization. scale bar=50 or 25 μm.

FIG. 5 Cell migration was increased following HG+Cyt insult, however HG+Cyt+Ton treatment partially prevented these changes. At 24 h, cells in the untreated group showed no significant change in scrape wound width (p=0.9789), while those treated with HG+Cyt exhibited significant scrape wound closure (p≤0.0001) relative to 0 h. HG+Cyt+Ton treatment led to a reduction in scrape wound closure, although a significant change was still observed (p=0.0015) relative to untreated cells. Statistical analysis was carried out using one-way ANOVA with Dunnett's multiple comparison test to compare treatments within a timepoint, and then to compare scrape wound closure within a treatment group across time. *comparison between treatment conditions at a given timepoint. **p≤0.01; ****p≤0.0001. +comparison with the 0 h timepoint within a given treatment group. ++p≤0.01; ++++p≤0.0001; n=10; dotted lines illustrate scrape wound edges; scale bar=50 μm.

FIG. 6. (a) FITC-dextran showed significantly higher permeability through a ARPE-19 cell monolayer following HG+Cyt insult in comparison to both untreated (p≤0.0001) and HG+Cyt+Ton treated cells (p≤0.0001). Statistical analysis was carried out using one-way ANOVA with Dunnett's multiple comparison test. ****p≤0.0001; n=3. (b) Trans-epithelial electrical resistance (TEER) was significantly reduced following HG+Cyt insult in comparison to untreated and HG+Cyt+Ton treated cells. From 24 h to 72 h, a significant treatment effect was observed, with the HG+Cyt group showing significantly lower TEER than both untreated (p=0.0292) and HG+Cyt+Ton (p=0.0015) treated groups. At 48 h the HG+Cyt insulted cells still showed significantly lower TEER than with HG+Cyt+Ton treatment (p=0.0029). By 72 h, TEER was again significantly lower in the HG+Cyt group relative to both untreated (p=0.0048) and HG+Cyt+Ton (p=0.0004) groups. Statistical analysis was carried out using two-way ANOVA with Dunnett's multiple comparison test. *p≤0.05; **p≤0.01; ***p≤0.001; n=3.

FIG. 7. (a) HG+Cyt induced TGF-β2 (p=0.0089) but not TGF-β1 release at 24 h. Tonabersat treatment (p=0.0057) prevented HG+Cyt induced TGF-β2 release back to untreated levels. (b) By 72 h, there were no significant differences between all groups in terms of both TGF-β1 and 2 levels. Statistical analysis was carried out using two-way ANOVA with Dunnett's multiple comparison test. *p≤0.05; **p≤0.01; ***p≤0.001; n=3.

### DETAILED DESCRIPTION

The process of EMT, whereby epithelial cells morph into mesenchymal cells, is a normal occurrence, necessary in embryonic morphogenesis to create diverse cell types with shared mesenchymal phenotype (Kalluri & Weinberg, 2009). Problems however arise when EMT occurs in other settings, such as cancer progression and tissue fibrosis, e.g., in the eye, the kidney and in other organs, and wherein tissue fibrosis develops following inflammation. In the eye, fibrosis of the retina results in both loss of retinal flexibility and gain of contractile properties, which can lead to loss of visual acuity as well as cause retinal detachment (Tamiya & Kaplan, 2016; Kroll et al., 2007). Recent research has implicated the NLRP3 inflammasome in EMT of human renal tubular and human bronchial epithelial cells, with knockdown of the NLRP3 inflammasome inhibiting EMT induction (Li et al., 2018; Song et al., 2018). However, the role of NRLP3 in regulating EMT in the retina, particularly RPE cells, remains unknown.

This patent shows that connexin hemichannels play a role in the EMT of RPE cells, further confirming the widespread influence of connexin 43 hemichannels in different pathologies. ARPE-19 cells were initially seen to change morphology following HG+Cyt insult, transforming from a classic cuboidal cobblestone monolayer to an elongated and spindle-like shape, reminiscent of fibroblastic cells present in fibrotic tissue. Progression between these two forms has already been well established as an indication of EMT (Tamiya & Kaplan, 2016; Wang et al., 2017; Hyttinen et al., 2019; Tamiya et al., 2010). As incubation time increased, the proportion of cells and extent of morphological change also increased. These morphological changes were supported by phenotypic adjustments following HG+Cyt insult; with decreased expression of the RPE specific marker, RPE65, countered by increased expression of the mesenchymal marker α-SMA. Additionally, α-SMA labelling showed that its cellular distribution became more elongated and striated. Again, these phenotypic changes are as expected for EMT, with Tamiya et al. (2010) previously demonstrating RPE65 expression to decrease with EMT progression, and acquisition of α-SMA expression frequently used as a marker of induced EMT (Wang et al., 2017; Song et al., 2018; Jing et al., 2019; Yang et al., 2020; Kobayashi et al., 2019).

These observed cellular changes translated to alterations in functional outcomes. DR-like insult (HG+Cyt) led to loss of cell membrane ZO-1 localisation, indicating a loss of cell-to-cell tight junctions. This loss in tight junctions allowed for greater cell mobility, which, combined with the changed cellular phenotype, contributed to an increase in cell migration, as indicated in the wound healing assay and as previously reported (Shukal et al., 2020). Results from FITC-dextran dye leak and TEER studies also confirmed that the barrier function of ARPE-19 cells exposed to HG+Cyt was lost, and if in vivo, would be no longer able to maintain its function as the outer BRB. Several studies have reported loss of RPE BRB integrity in disease, including DR, AMD and uveitis (Cunha-Vaz, 2004, 2009; Klaassen et al., 2013). Furthermore, EMT has been described as a key pathology resulting in loss of RPE functionality (Che et al., 2016; Li et al., 2020). Hyttinen et al. (2019) discussed the links between EMT and AMD, a condition in which RPE fibrosis is a main characteristic, while also suggesting that EMT may cause the RPE destruction seen in the “terminal” phase of AMD through increased cell migration combined with cell detachment. Finally, in PVR, the acquired contractile properties of transitioned cells, which form epiretinal membranes, leads to retinal folds and traction mediated retinal detachments (Tamiya & Kaplan, 2016). For DR and wet AMD, anti-vascular endothelial growth factors (anti-VEGFs) are currently the gold standard treatment, but this does little to effect the underlying cause of disease, nor are valuable in the early stages of disease (Campochiaro et al., 2016; Kovach et al., 2012; Dhoot & Avery, 2016).

In the Examples, addition of the connexin hemichannel inhibitor tonabersat prevented the phenotypic changes otherwise observed following HG+Cyt insult, showing Cx43 hemichannels to be crucial to EMT in RPE cells. Our findings present a new therapeutic target, as well as the potential to treat EMT-related ocular diseases, for example. Importantly, they also demonstrate the utility of connexin hemichannel inhibitor treatment in the early stages of EMT-related ocular diseases, by countering the underlying cause.

The Examples demonstrate that insult of epithelial cells with HG+Cyt using RPE cells induces EMT. Further, blocking connexin hemichannels attenuates EMT, demonstrating that hemichannels play an important role in facilitating the process and providing another therapeutic target for diseases underlain by EMT. These findings offer opportunities for those with health conditions such as diabetes, where eye diseases, now frequently linked to EMT, are both common and highly debilitating.

We have discovered that increased connexin43 hemichannel opening is associated with EMT activation, and in a range of pathologies including ocular disorders. We have discovered the utility of clinically safe doses of connexin hemichannel blockers, such as orally-delivered small molecule connexin hemichannel blockers, including Xiflam, in the inhibition of EMT. We have discovered that hemichannel blockers can be used to improve EMT-related chronic retinal diseases.

### Definitions

The term “comprising,” which is synonymous with “including,” “containing,” or “characterized by,” is inclusive or open-ended and does not exclude additional, unrecited elements or ingredients from the medicament (or steps, in the case of a method). The phrase “consisting of” excludes any element, step, or ingredient not specified in the medicament (or steps, in the case of a method). The phrase “consisting essentially of” refers to the specified materials and those that do not materially affect the basic and novel characteristics of the medicament (or steps, in the case of a method). The basic and novel characteristics of the inventions are described throughout the specification, and include the ability of medicaments and methods of the invention to block or modulate connexin gap junction hemichannels and to modulate or inhibit EMT and/or EndMT, as the case may be. Material changes in the basic and novel characteristics of the inventions, including the medicaments and methods described herein, include an unwanted or clinically undesirable, detrimental, disadvantageous or adverse diminution of hemichannel modulation and/or modulation or inhibition of EMT and/or EndMT. In one embodiment, the medicament will comprise, consist essentially of, or consist of a connexin 43 hemichannel blocker, for example, a small molecule connexin 43 hemichannel blocker.

As used here, the term “about” a value or parameter refers to its meaning as understood in the art and includes embodiments that are directed to that value or parameter per se. For example, description referring to “about X” includes description of “X.” For example, the term “about 5 mg” of a weight value in a dosage refers to +/−0.5 degrees of the weight value.

A “small molecule” is defined herein to have a molecular weight below about 600 to 900 daltons, and is generally an organic compound. A small molecule can be an active agent of a hemichannel blocker prodrug. In one embodiment, the small molecule is below 600 daltons. In another embodiment, the small molecule is below 900 daltons.

As used herein, “treatment” (and grammatical variations thereof such as “treat” or “treating”) refers to clinical intervention to alter the natural course of the individual, tissue or cell being treated, and can be performed either for prophylaxis or during clinical pathology. Desirable effects of treatment include, but are not limited to, preventing occurrence or recurrence of a disease, disorder or condition, alleviation of signs or symptoms, diminishment of any direct or indirect pathological consequences of the disease, decreasing the rate of disease progression, amelioration or palliation of the disease state, and remission or improved prognosis. In some embodiments, compounds, methods and compositions of the invention can be used to delay development of a disease, disorder or condition, or to slow the progression of an EMT-related disease, disorder or condition. The term does not necessarily imply that a subject is treated until total recovery. Accordingly, “treatment” includes reducing, alleviating or ameliorating the symptoms or severity of a particular disease, disorder or condition or preventing or otherwise reducing the risk of developing a particular disease, disorder or condition. It may also include maintaining or promoting a complete or partial state of remission of a condition.

“Treatment” as used herein also includes inhibiting EMT activity in a subject, following administration of a hemichannel blocker. A preferred hemichannel blocker is Xiflam. A preferred route of the administration is oral.

The term “treating” a disease, condition or disorders or the like, may refer to preventing, slowing, reducing, decreasing and, notably, to stopping and reversing an EMT-related disorder, disease or condition.

In other embodiments, the retina is protected using the compounds and methods described herein, as shown in the Examples, which is important in chronic retinal diseases, including age-related macular degeneration, where the protective effects of the invention also find utility.

The term “preventing” means preventing in whole or in part, or ameliorating, or controlling.

As used herein, “effective amount” refers to an amount effective, at dosages and for periods of time necessary, to achieve the desired therapeutic or prophylactic result. For example, and not by way of limitation, an “effective amount” can refer to an amount of a compound or composition, disclosed herein, that is able to treat the signs and/or symptoms of a disease, disorder or condition that involve pathological or otherwise unwanted EMT activity, or to an amount of a hemichannel compound or composition that is able to beneficially maintain normal or near-normal EMT function.

As used herein, “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is preferably also one in which any toxic or detrimental effects of the substance/molecule, agonist or antagonist may be outweighed by the therapeutically beneficial effects. A therapeutically effective amount of a hemichannel blocker will beneficially inhibit EMT activity in a subject.

As used herein, “prophylactically effective amount” refers to an amount effective, at dosages and for periods of time necessary, to achieve a desired prophylactic result, typically inhibition of unwanted EMT activity. Typically, but not necessarily, the prophylactically effective amount will be less than the therapeutically effective amount.

The term “pharmaceutical formulation” refers to a preparation which is in such form as to permit the biological activity of an active ingredient contained therein, e.g., a hemichannel blocker, to be effective, and which does not contain additional components that are unacceptably toxic to a subject to whom the formulation would be administered.

A “pharmaceutically acceptable carrier,” as used herein, refers to an ingredient in a pharmaceutical formulation, other than an active ingredient, which can be safely administered to a subject. A pharmaceutically acceptable carrier includes, but is not limited to, buffers, excipients, stabilizers, and preservatives.

As used herein, the term “subject” or the like, including “individual,” and “patient”, all of which may be used interchangeably herein, refers to any mammal, including humans, domestic and farm animals, and zoo, wild animal park, sports, or pet animals, such as dogs, horses, cats, sheep, pigs, cows, etc. The preferred mammal is a human, including adults, children, and the elderly. Preferred sports animals are horses and dogs. Preferred pet animals are dogs and cats. In certain embodiments, the subject, individual or patient is a human.

EMT may be measure and monitored and visualized in vivo using art known methods. EMT is characterized by a loss of epithelial cell markers, such as cytokeratins and E-cadherin, followed by an upregulation in the expression of mesenchymal cell markers, such as N-cadherin, vimentin and fibronectin. Epithelial and mesenchymal cell marker expression changes lead to a reduction in adhesion between the transitioning cell and adjacent epithelial cells, and an increase in the secretion of enzymes that degrade the extracellular matrix. Collectively, this results in epithelial cells losing apical-basal cell polarity, reorganizing their cytoskeleton, and reprogramming gene expression. In cancer, this enables the development of an invasive phenotype in cancer metastasis. EMT may be measured using a number of methods. See, e.g., Busch, E L, et al. Evaluating markers of epithelial-mesenchymal transition to identify cancer patients at risk for metastatic disease Clin Exp Metastasis 2016 January; 33(1):53-62 (measurement of EMT markers in primary tumor specimens); Song J., et al., Epithelial-mesenchymal transition markers screened in a cell-based model and validated in lung adenocarcinoma BMC Cancer Volume 19, Article number: 680 (2019); Michael Zeisberg and Eric G. Neilson, Biomarkers for epithelial-mesenchymal transitions J Clin Invest. 2009; 119(6):1429-1437; Epithelial-Mesenchymal Transition (EMT) Markers https://www.novusbio.com/antibody-news/antibodies/antibodies-for-epithelial-mesenchymal-transition-emt-marker Aug. 18, 2016-14:15. EMT may be visualized and monitored using known techniques, such as the EMT imaging system described in Ieda, T., et al., Visualization of epithelial-mesenchymal transition in an inflammatory microenvironment—colorectal cancer network Sci Rep 9, 16378 (2019) (In vivo spatiotemporal visualization of CRC cells undergoing EMT using a fluorescence-guided EMT imaging system in which the mesenchymal vimentin promoter drives red fluorescent protein (RFP) expression); Maie, J. et al., Visualizing Epithelial-Mesenchymal Transition Using the Chromobody Technology Cancer Res; 76(19); 5592-6 (discussing the chromobody technology and visualization of EMT-related processes in living systems). In “The basics of epithelial-mesenchymal transition,” Kalluri and Weinberg describe the changes epithelial cell undergo until they become mesenchymal, which range from the activation and deactivation of transcription factors and expression of specific mRNAs, to changes in the expression and structure of cytoskeletal and cell-surface proteins (Kalluri & Weinberg, 2009).

In the eye, EMT can be visualized, measured and monitored using optical coherence tomography (OCT), a simple, non-invasive imaging test. Ocular EMT can be measured directly by visualizing changes in cell morphology. In the cornea, for example, Iridocorneal Endothelial Syndrome, results in the corneal endothelium having a “hammered silver” or “beaten bronze” appearance in ICE syndrome patients, similar to corneal guttae seen in Fuchs Corneal Endothelial Dystrophy. On a pathological level, the normal endothelial cells have been replaced with a more epithelial-like cell with migratory characteristics. This can be observed using in vivo confocal microscopy (see for example Grupcheva C N et al., In vivo confocal microscopic characteristics of iridocorneal endothelial syndrome. Clin Exp Ophthalmol. 2004; 32:275-283. In the retina, disruption to RPE function has been implicated in multiple ocular diseases including diabetic retinopathy (DR), age-related macular degeneration (AMD) and proliferative vitreoretinopathy (PVR). Changes to the retinal pigment epithelium are visualized directly using fundus imaging to show loss of pigmented epithelium as cells undergo EMT, and using OCT to measure retinal pigment epithelium thickness and integrity, including increased reflectivity resulting from RPE hyperplasia indicating EMT has/is occurring.

As used herein, the term “hemichannel” is a part of a gap junction (two hemichannels or connexons connect across an intercellular space between adjacent cells to form a gap junction) and is comprised of a number of connexin proteins, typically homologous or heterologous, i.e., homo- or hetero-meric hexamers of connexin proteins, that form the pore for a gap junction between the cytoplasm of two adjacent cells. The hemichannel is supplied by a cell on one side of the junction, with two hemichannels from opposing cells normally coming together to form the complete intercellular hemichannel. However, in some cells, and in cells under some circumstances, the hemichannel itself is active as a conduit between the cytoplasm and the extracellular space allowing the transfer of ions and small molecules.

Compounds of Formula I, for example Xiflam, and/or an analogue or pro-drug of any of the foregoing compounds, can modulate the function and/or activity of hemichannels, preferably those comprising any type of connexin protein. Accordingly, reference to “hemichannel” should be taken broadly to include a hemichannel comprising, consisting essentially of, or consisting of any one or more of a number of different connexin proteins, unless the context requires otherwise. However, by way of example, a hemichannel may comprise one or more of any connexin, including those referred to specifically above. In one embodiment, a hemichannel consists of one of the aforementioned connexins. In one embodiment, a hemichannel comprises one or more of connexin 36, 37, 40, 43, 45 and 57. In one embodiment, a hemichannel consists of one of connexin 37, 40, or 43. In one embodiment, the hemichannel is a connexin 43 hemichannel. In one embodiment, a hemichannel is retinal hemichannel. In one embodiment, hemichannel is choroidal hemichannel. In one embodiment, the hemichannel is a vascular hemichannel. In one embodiment, a hemichannel is a connexin hemichannel found in vascular endothelial cells. In one particular embodiment, a hemichannel comprises one or more of connexin 30, 37 and connexin 43. In one particular embodiment, a hemichannel consists of connexin 30. In one particular embodiment, a hemichannel consists of connexin 37. In one particular embodiment, a hemichannel consists of connexin 43. In one embodiment, the hemichannel comprises one or more connexins excluding connexin 26. In one embodiment, the composition can include or exclude a hemichannel blocker of any connexin, including the foregoing.

Hemichannels and hemichannels may be present in cells of any type. Accordingly, reference to a “hemichannel” or a “hemichannel” should be taken to include reference to a hemichannel or hemichannel present in any epithelial or endothelial cell type, and which will be a target for inhibition of EMT or EndMT. In one embodiment of the invention, the hemichannel or hemichannel is present in a cell in an organ, or in a cancer or tumor. In one embodiment, the hemichannel is a vascular hemichannel. In one embodiment, the hemichannel is a connexin hemichannel found in vascular endothelial cells and/or in ocular epithelial or endothelial cells.

As used herein, “modulation of a hemichannel” is the modulation of one or more functions and/or activities of a hemichannel, typically, the flow of molecules between cells through a hemichannel Such functions and activities include, for example, the flow of molecules from the extracellular space or environment through a hemichannel into a cell, and/or the flow of molecules through a hemichannel from the intracellular space or environment of a cell into the extracellular space or environment. Compounds useful for modulation of a hemichannel may be referred to as “hemichannel modulators” or “hemichannel inhibitors.” All aspects of the inventions and methods described herein may be accomplished by modulation of a hemichannel to disrupt its activity, including inhibiting or blocking hemichannel opening and/or release of ATP, for example. Modulators or inhibitors of a connexin hemichannel are also referred to herein as “anti-hemichannel compounds,” including, for example, anti-connexin 43 hemichannel compounds.

Modulation of the function of a hemichannel may occur by any means. However, by way of example only, modulation may occur by one or more of: inducing or promoting closure of a hemichannel; preventing, blocking, inhibiting or decreasing hemichannel opening; triggering, inducing or promoting cellular internalization of a hemichannel and/or gap junction. Use of the words such as “blocking”, “inhibiting”, “preventing”, “decreasing” and “antagonizing”, and the like, may not be taken to imply complete blocking, inhibition, prevention, or antagonism, although this may be preferred, and shall be taken to include partial blocking, inhibition, prevention or antagonism to at least reduce the function or activity of a hemichannel and/or hemichannel. Similarly, “inducing” or “promoting” should not be taken to imply complete internalization of a hemichannel (or group of hemichannels) and should be taken to include partial internalization to at least reduce the function or activity of a hemichannel.

As used herein, the terms “anti-hemichannel compound” and “hemichannel blocker” is a compound that interferes with the passage of molecules through a connexin hemichannel. An anti-hemichannel compound or hemichannel blocker can block or decrease hemichannel opening, block or reduce the release of molecules through a hemichannel to an extracellular space, and/or block or reduce the entry of molecules through a hemichannel into an intracellular space. Anti-hemichannel compound and hemichannel blockers include compounds that fully or partially block hemichannel leak or the passage of molecules to or from the extracellular space. Anti-hemichannel compound and hemichannel blockers also include compounds that decrease the open probability of a hemichannel Open probability is a measure of the percentage of time a channel remains open versus being closed (reviewed in Goldberg G S, et al., Selective permeability of gap junction channels Biochimica et Biophysica Acta 1662 (2004) 96-101). Anti-hemichannel compound and hemichannel blockers include hemichannel modulators. Anti-hemichannel compound and hemichannel blockers may interfere directly, or directly, with the passage of molecules through a connexin hemichannel. All aspects of the inventions and methods described herein may be accomplished by blocking a hemichannel, or decreasing the open probability of a hemichannel, for example, as described herein. In one embodiment, the connexin hemichannel is a connexin 43 hemichannel, and/or other vascular connexin hemichannel.

As used herein, the terms “inhibit EMT” and “inhibit EndMT” and the like, refer to lowering, diminishing or downregulating epithelial-mesenchymal transition or endothelial-mesenchymal transition, as the case may be. In some embodiments of the invention, retinal pigment epithelium, retinal vascular endothelium, EMT and/or EndMt are returned to a normal or pre-disease state.

The terms “peptide,” “peptidomimetic” and “mimetic” include synthetic or genetically engineered chemical compounds that may have substantially the same structural and functional characteristics of protein regions which they mimic. In the case of connexin hemichannels, these may mimic, for example, the extracellular loops of hemichannel connexins.

The patent describes new methods to EMT- and/or EndMT-related diseases, disorders or conditions which can be improved by the methods of the invention.

The instant inventions provide, inter alia, methods for inhibition of EMT and/or EndMT activity by administration of a hemichannel blocker, such as compounds of Formula I, for example Xiflam, or compounds of Formula II, and/or an analogue or pro-drug of any of the foregoing compounds, for the treatment of a disease, disorder or condition characterized in whole or in part by pathological or otherwise unwanted EMT and/or EndMT activity.

In some embodiments, this invention features the use of compounds of Formula I, for example Xiflam, or compounds of Formula II, and/or an analogue or pro-drug of any of the foregoing compounds to directly and immediately block Cx43 hemichannels and to cause the inhibition of EMT and/or EndMT. Some exemplary doses are in the range of about 1.0 to about 7.0 mg/kg, including, for example, from 1.0 to 3.0 mg/kg, or from 3.0 to 4.0 mg/kg and from 4.0 to 5.0 mg/kg, or 1.1 to 1.5 mg/kg. Some exemplary daily or other periodic dose amounts range from about 10-250 mg per dose, including, for example, from about 80-160 mg per dose from about 160-240 mg per dose, from about 240-300 mg per dose and from about 300-500 mg per dose, including doses of 80, 150, 250, and 500 mg per dose.

### Connexins

In various embodiments, the hemichannel being modulated is any connexin hemichannel, and may include or exclude a connexin 26 (Cx26) hemichannel. In certain embodiments, the hemichannel being modulated is a connexin 36 (Cx36) hemichannel, a connexin 37 (Cx37) hemichannel, a connexin 40 (Cx40) hemichannel, a connexin 43 (Cx43) hemichannel, a connexin 45 (Cx45) hemichannel, and/or a connexin 57 (Cx57) hemichannel. In one embodiment, the hemichannel being modulated comprises one or more of a Cx36, Cx37, Cx40, Cx43, Cx45 and/or Cx57 protein. In one particular embodiment, the hemichannel and/or hemichannel being modulated is a Cx37 and/or Cx40 and/or Cx43 hemichannel. In one particular embodiment, the hemichannel and/or hemichannel being modulated is a Cx30 and/or Cx43 and/or Cx45 hemichannel. In one particular embodiment, the hemichannel and/or hemichannel being modulated is a Cx36, Cx37, Cx43 and/or Cx45 hemichannel.

In some embodiments, the hemichannel being modulated can include or exclude any of the foregoing connexin proteins. In some aspects, the hemichannel blocker is a blocker of a Cx43 hemichannel, a Cx40 hemichannel and/or a Cx45 hemichannel. In certain preferred embodiments, the hemichannel blocker is an epithelial and/or endothelial cell connexin 43 hemichannel blocker. The pharmaceutical compositions of this invention for any of the uses featured herein may also comprise a hemichannel blocker that may inhibit or block any of the noted connexin hemichannels (including homologous and heterologous hemichannels). In some embodiments the hemichannel being modulated can include or exclude any of the foregoing connexin hemichannels, or can be a heteromeric hemichannel.

The hemichannel blocker used in any of the administration, co-administrations, compositions, kits or methods of treatment of this invention is a Cx43 hemichannel blocker, in one embodiment. Other embodiments include Cx45 hemichannel blockers, Cx30 hemichannel blockers, Cx37 hemichannel blockers, Cx40 hemichannel blockers, and blockers of one or another of the connexin hemichannel or a hemichannel comprising noted above or herein, or consisting essentially of, or consisting of any other connexins noted above or herein. Some embodiments may include or exclude any of the foregoing connexins or hemichannels, or others noted in this patent. In various embodiments, by way of example, the hemichannel being modulated comprises one or more of connexin 36, connexin 37, connexin 40, connexin 43, connexin 45, connexin 57, connexin 59 and/or connexin 62.

In one embodiment, particularly as it relates to the retina, the hemichannel being modulated comprises one or more of a Cx36, Cx37, Cx40, Cx43, Cx45 or Cx57 protein. Targeted hemichannel connexins include one or more of selected hemichannel connexins in blood vessels (e.g., Cx37, Cx40 or Cx43), as well as hemichannel connexins in neuroepithelial cells, such as astroglial cells (e.g., Cx43), amacrine cells (e.g., Cx36, Cx45), bipolar cells (e.g., Cx36, Cx45), the outer and inner plexiform layer, the ganglion cell layer (e.g., Cx36, Cx45), cone photoreceptors and retinal endothelial cells, and other retinal neurons, for example. In some embodiments, Cx36 and Cx43 hemichannels are targeted. In one particular embodiment, the hemichannel and/or hemichannel being modulated comprises Cx43. In one embodiment, hemichannels comprising connexins in the cells of the outer plexiform layer are targeted (e.g., Cx43), where methods of the invention can stop and reverse OPL thinning and rescue the OPL.

In other embodiments, particularly those relating to the choroid or blood vessels of the retina, the hemichannel being modulated may preferentially comprise one or more of a Cx37, Cx40 or Cx43 protein. In one particular embodiment, the hemichannel and/or hemichannel being modulated comprises Cx43. In one embodiment, hemichannels comprising vessel connexins in cells of the outer choroid, also known as Haller's layer, which is composed of large caliber, non-fenestrated vessels, are targeted. In another embodiment, hemichannels comprising vessel and endothelial cell connexins in cells of the inner choroid, also known as Sattler's layer, which is composed of significantly smaller vessels, are targeted. In another embodiment, hemichannels comprising connexins in cells of the outer and inner choroid are targeted. In another embodiment, hemichannels comprising connexins in capillaries of the choriocapillaris are targeted. In one embodiment, hemichannel vessel connexins targeted in methods of the invention include hemichannel connexins in pericytes and connexins in vascular smooth muscle and endothelial cells. In another embodiment, hemichannel vessel connexins targeted in methods of the invention include hemichannels in pericytes and connexins in endothelial cells, for example, in the microcapillaries. Cx43 hemichannels are a preferred target of the invention.

### Small Molecule Hemichannel Blockers

Examples of hemichannel blockers include small molecule hemichannel blockers, e.g., Xiflam (tonabersat). The structure of tonabersat (also shown in PubChem, DrugBank, and MedChemExpress) is:

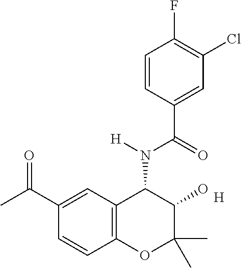

Other chemical names for tonabersat are found in PubChem (N-[(3S,4S)-6-acetyl-3-hydroxy-2,2-dimethyl-3,4-dihydrochromen-4-yl]-3-chloro-4-fluorobenzamide) , DrugBank (N-[(3S,4S)-6-acetyl-3-hydroxy-2,2-dimethyl-3,4-dihydro-2H-1-benzopyran-4-yl]-3-chloro-4-fluorobenzamide) and Chemical Book (N-((3S,4S)-6-Acetyl-3-hydroxy-2,2-dimethylchroman-4-yl)-3-chloro-4-fluorobenzamide; or 2H-Benzo(B)pyran-3-ol, 6-acetyl-4-(3-chloro-4-fluorobenzoylamino)-3,4-dihydro-2,2-dimethyl-; or N-[(3S,4S)-6-Acetyl-3,4-dihydro-3-hydroxy-2,2-dimethyl-2H-1-benzopyran-4-yl]-3-chloro-4-fluoro-benzamide).

In some embodiments, the hemichannel blocker is a small molecule other than Xiflam, for example, a hemichannel blocker described in Formula I or Formula II in US Pat. App. Publication No. 20160177298, filed in the name of Colin Green, et al., the disclosure of which is hereby incorporated in its entirety by this reference, as noted above. Various preferred embodiments include use of a small molecule that blocks or ameliorates or otherwise antagonizes or inhibits hemichannel opening, to treat EMT- and/or EndMT-related diseases, disorders and conditions, including those diseases, disorders and conditions described or referenced herein. In various embodiments, the small molecule that blocks or ameliorates or inhibits hemichannel opening is a prodrug of Xiflam or an analogue thereof.

In some embodiments, this invention features the use of small molecule hemichannel blockers including, for example, compounds of Formula I, such as Xiflam, and/or an analogue or pro-drug of any of the foregoing compounds to block Cx43 hemichannels, for example, for the treatment of an EMT- and/or EndMT-related disease, disorder or condition.

By way of example, the hemichannel blocker Xiflam (tonabersat) may be known by the IUPAC name N-[(3S,4S)-6-acetyl-3-hydroxy-2,2-dimethyl-3,4-dihydrochromen-4-yl]-3-chloro-4-fluorobenzamide or (3S-cis)-N-(6-acetyl-3,4-dihydro-3-hydroxy-2,2-(dimethyl-d6)-2H-1-benzopyran-4-yl)-3-chloro-4-fluorobenzamide

Another useful compound is boldine, an alkaloid of the aporphine class found in the boldo tree and in Lindera aggregata.

In one embodiment, Xiflam and/or an analogue or prodrug thereof is chosen from the group of compounds having the Formula I:

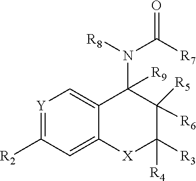

wherein
Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—,
—CO—, —CH₂—, CH(OH), SO₂, SO, CH₂—O, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆alkyl or by C₂₋₇ alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C₁₋₆ alkyl amino, amino-C₁₋₆ alkyl, hydroxy-C₁₋₆ alkyl, halo-C₁₋₆ alkyl, C₁₋₆ acyloxy-C₁₋₆ alkyl, C₁₋₆alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆ alkyl.

In some embodiments, this invention features the use of small molecule hemichannel blockers including, for example, compounds of Formula II, and/or an analogue or pro-drug of any of the foregoing compounds to block Cx43 hemichannels, for example, for the inhibition of EMT and/or EndMT activity.

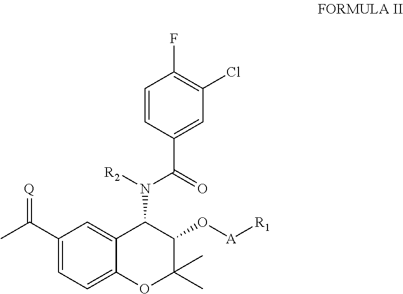

wherein
Q is O or an oxime of formula ═NHOR₄₃, wherein R₄₃ is
(i) selected from H, C₁₋₄ fluoroalkyl or optionally substituted C₁₋₄ alkyl, or
(ii) -A₃₀₀-R₃₀₀ wherein
A₃₀₀ is a direct bond, —C(O)O*—, —C(R₃)(R₄)O*—, —C(O)O—C(R₃)(R₄)O*—, or —C(R₃)(R₄)OC(O)O*— wherein the atom marked* is directly connected to R₃₀₀,
R₃ and R₄ are selected independently from H, fluoro, C₁₋₄ alkyl, or C₁₋₄ fluoroalkyl, or
R₃ and R₄ together with the atom to which they are attached form a cyclopropyl group,
R₃₀₀ is selected from groups [1], [2], [2A], [3], [4], [5] or [6];
R₂ is H or B—R₂₁,
A is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₁, R₃ and R₄ are selected independently from H, fluoro, C₁₋₄ alkyl, or C₁₋₄ fluoroalkyl, or R₃ and R₄ together with the atom to which they are attached form a cyclopropyl group,
R₁ is selected from groups [1], [2], [2A], [3], [4], [5] and [6] wherein the atom marked ** is directly connected to A:

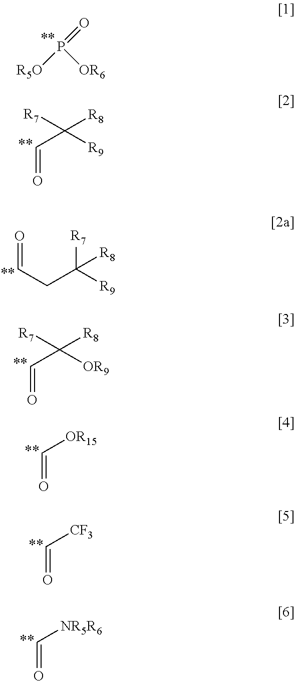

R₅ and R₆ are each independently selected from H, C₁₋₄ alkyl, C₁₋₄ fluoroalkyl, and benzyl;
R₇ is independently selected from H, C₁₋₄ alkyl, and C₁₋₄ fluoroalkyl;
R₈ is selected from:
(i) H, C₁₋₄ alkyl, or C₁₋₄ fluoroalkyl, or
(ii) the side chain of a natural or unnatural alpha-amino acid, or a peptidomimetic or other peptide as described herein, or
(iii) biotin or chemically linked to biotin;
R₉ is selected from H, —N(R₁₁)(R₁₂), or —N⁺(R₁₁)(R₁₂)(R₁₃)X⁻, or —N(R₁₁)C(O)R₁₄ wherein R₁₁, R₁₂, and R₁₃ are independently selected from H, C₁₋₄ alkyl, or C₁₋₄ fluoroalkyl,
R₁₄ is H, C₁₋₄ alkyl, or C₁₋₄ fluoroalkyl,
R₁₅ is independently selected from C₁₋₄ alkyl and C₁₋₄ fluoroalkyl, and
X⁻ is a pharmaceutically acceptable anion.
In some embodiments, Q is O.

For any of the Markush groups set forth above, that group can include or exclude any of the species listed for that group. Hemichannel blockers for use in methods of the invention may include or exclude any of these compounds.

In another embodiment, the analogue of Formula I is the compound carabersat (N-[(3R,4S)-6-acetyl-3-hydroxy-2,2-dimethyl-3,4-dihydrochromen-4-yl]-4-fluorobenzamide) or trans-(+)-6-acetyl-4-(S)-(4-fluorobenzoylamino)-3,4-dihydro-2,2-dimethyl-2H-1-benzo[b]pyran-3R-ol, hemihydrate.

In certain embodiments, Xiflam and/or an analogue thereof are in the form of a free base or a pharmaceutically acceptable salt. In other embodiments, one or more polymorph, one or more isomer, and/or one or more solvate of Xiflam and/or an analogue thereof may be used.

Other various small molecules have been reported to useful in inhibiting hemichannel activity. See Green et al., US Pat. App. Publication No. 20160177298, Formula II; Savory, et al., US Pat. App. Publication No. 20160318891; and Savory, et al., US Pat. App. Publication No. 20160318892, all of which are incorporated in their entireties by reference, as noted above. The hemichannel blockers for use in methods of the invention may include or exclude any of these compounds.

In one aspect, the invention relates to the use of pharmaceutical compositions, alone or within kits, packages or other articles of manufacture, in methods for treating diseases, disorders, or conditions noted herein, as well as those characterized by pathological or otherwise unwanted EMT and/or EndMT activity. In some aspects, the hemichannel blocker is a connexin 43 hemichannel blocker. Blockers of other connexin hemichannels are within the invention, as noted.

In some embodiments “promoiety” refers to a species acting as a protecting group which masks a functional group within an active agent, thereby converting the active agent into a pro-drug. Typically, the promoiety will be attached to the drug via bond(s) that are cleaved by enzymatic or non-enzymatic means in vivo, thereby converting the pro-drug into its active form. In some embodiments the promoiety may also be an active agent. In some embodiments the promoiety may be bound to a hemichannel blocker molecule, peptide, antibody or antibody fragment. In some embodiments the promoiety may be bound to any of a peptide or peptidomimetic or small molecule or other organic hemichannel blocker, for example. In some embodiments the promoiety may be bound to a compound of Formula I. In some embodiments the pro-drug may be another hemichannel compound, e.g., a compound described in Green et al., US Pat. App. Publication No. 20160177298; Savory, et al., US Pat. App. Publication No. 20160318891; or Savory, et al., US Pat. App. Publication No. 20160318892.

### Methods of Treatment

One method of the invention comprises the steps of (1) identifying a subject with an EMT-related disease, disorder or condition, (2) administering a therapeutically effect amount of a connexin hemichannel inhibitor to the subject and, optionally, (3) measuring or visualizing EMT activity the subject. In one embodiment, the EMT activity is measured or visualized and the dose is maintained or adjusted. In one embodiment of the method, step (1) is not required because the subject is already known to have an EMT-related disease.

In one embodiment, the disease, disorder or condition is an EndMT-related disease, disorder or condition, and EndMT activity is optionally measured. In one embodiment, the EndMT activity is measured or visualized and the dose is maintained or adjusted.

In one embodiment, EMT and is lessened, inhibited or otherwise attenuated.

In one embodiment, EndMT is lessened, inhibited or otherwise attenuated.

In one embodiment of the method, the connexin hemichannel inhibitor is a connexin 43 hemichannel inhibitor.

In one embodiment of the method, the connexin 43 hemichannel inhibitor is a small molecule connexin 43 hemichannel inhibitor.

In another embodiment of the method, the connexin hemichannel inhibitor is an anti-connexin 43 hemichannel peptide or peptidomimetic that inhibits or blocks connexin 43 hemichannel opening or activity.

In one embodiment of the method, the connexin 43 hemichannel inhibitor is tonabersat. In another embodiment of the method, the connexin 43 hemichannel inhibitor is carabersat.

In one embodiment of the method, the EMT-related disease, disorder or condition in the subject is characterized by EMT dysregulation. In another embodiment of the method, the EndMT-related disease, disorder or condition in the subject is characterized by EndMT dysregulation.

In one embodiment of the method, the EMT-related disease, disorder or condition in the subject is characterized in whole or in part by pathological or otherwise unwanted EMT activity. In one embodiment of the method, the EndMT-related disease, disorder or condition in the subject is characterized in whole or in part by pathological or otherwise unwanted EndMT activity. In one embodiment of the method, the EMT- or EndMT-related disease, disorder or condition in the subject is diabetic retinopathy, age-related macular degeneration or proliferative vitreoretinopathy.

In another embodiment of the method, the EMT- or EndMT-related disease, disorder or condition in the subject is a retinal or other disorder characterized by a pathological or otherwise unwanted level of EMT activity.

In another embodiment of the method, the EMT- or EndMT-related disease, disorder or condition in the subject is a fibrosis disorder. In one embodiment, the EMT and/or EndMT modulation or inhibition treats an ocular fibrosis disorder.

In another embodiment of the method, the EMT- or EndMT-related disease, disorder or condition in the subject is a cancer. In some embodiments the EMT modulation or inhibition (or EndMT modulation or inhibition) using, for example, compounds and methods to modulate connexin hemichannels, including connexin 43 hemichannels, treats kidney fibrosis. In some embodiments, the EMT modulation or inhibition (or EndMT modulation or inhibition) treats renal failure or chronic renal failure. In some embodiments, the EMT modulation or inhibition (or EndMT modulation or inhibition) treats EMT in renal epithelial cells following kidney injury. In some embodiments the EMT modulation or inhibition (or EndMT modulation or inhibition) treats fibrosis in organs other than the eye and kidney. In some embodiments, the EMT modulation or inhibition (or EndMT modulation or inhibition) treats fibrosis following inflammation. In some embodiments, the EMT and/or EndMT modulation or inhibition treats EMT or EndMT in renal epithelial cells following kidney injury. In some methods the EMT modulation or inhibition (or EndMT modulation or inhibition) treats any fibrosis/fibrotic disorder. In one embodiment, the EMT modulation or inhibition treats an ocular fibrosis disorder. Epithelial-mesenchymal transition has become widely accepted as a mechanism by which injured renal tubular cells transform into mesenchymal cells that contribute to the development of fibrosis in the kidney and in chronic renal failure, and in some embodiments the EMT modulation or inhibition (or EndMT modulation or inhibition) using, for example, compounds and methods to modulate connexin hemichannels, including connexin 43 hemichannels, treats renal fibrosis. In some embodiments, the EMT modulation or inhibition (or EndMT modulation or inhibition) treats renal failure or chronic renal failure. In some embodiments, the EMT modulation or inhibition (or EndMT modulation or inhibition) treats EMT and/or EndMT in renal epithelial cells following kidney injury. In some embodiments, the EMT modulation or inhibition (or EndMT modulation or inhibition) treats fibrosis following inflammation. In some embodiments, the EMT modulation or inhibition (or EndMT modulation or inhibition) treats any fibrotic disorder. As used herein, fibrotic disorders include and any disease, disorder or condition where epithelial cells are induced to acquire a myofibroblast phenotype and ultimately a fibrotic phenotype. EMT- and EndMT-related fibrotic disorders treatable with compounds and methods of the invention include, for example, pulmonary (lung) fibrosis, kidney fibrosis, idiopathic pulmonary fibrosis, liver fibrosis, intestinal fibrosis, ocular fibrosis, adipose tissue fibrosis, cardiac and other organ fibroses, as well as scleroderma. In some embodiments, fibrotic conditions leading to the most common causes of hepatic fibrosis, namely, hepatitis B and C, nonalcoholic steatohepatitis, and alcohol abuse, are treatable with compounds and methods of the invention. Subjects with hepatic activity grades ranging from A1 to A3 and/or fibrosis stages ranging from F1 to P3 may be treated with compounds and methods of the invention, for example.

### Chemical Delivery Modification

Hemichannel blockers useful in the present invention can also be formulated into microparticle (microspheres, Mps) or nanoparticle (nanospheres, Nps) formulations, or both, as well as liposomes or implants. Particulate drug delivery systems include nanoparticles (1 to 999 nm) and microparticles (1 to 1,000 μm), which are further categorized as nanospheres and microspheres and nanocapsules and microcaps. In nanocapsules and microcapsules, the drug particles or droplets are entrapped in a polymeric membrane. Particulate systems have the advantage of delivery by injection, and their size and polymer composition influence markedly their biological behavior in vivo. Microspheres can remain in the vitreous for much longer periods of time than nanospheres, therefore, microparticles act like a reservoir after injection. Nanoparticles diffuse rapidly and are internalized in tissues and cells.

### Assessing Hemichannel Blocker Activity

Various methods may be used for assessing the activity or efficacy of hemichannel blockers. In one aspect of the invention, the effects of hemichannel blocker treatment in a subject is evaluated or monitored using techniques to evaluate EMT and/or EndMT activity, as described herein, by way of example.

The activity of hemichannel blockers may also be evaluated using certain biological assays. Effects of known or candidate hemichannel blockers on molecular motility can be identified, evaluated, or screened for using the methods described in the Examples below, or other art-known or equivalent methods for determining the passage of compounds through connexin hemichannels. Various methods are known in the art, including dye transfer experiments, for example, transfer of molecules labelled with a detectable marker, as well as the transmembrane passage of small fluorescent permeability tracers, which has been widely used to study the functional state of hemichannels. See, for example, Schlaper, K A, et al. Currently Used Methods for Identification and Characterization of Hemichannels. Cell Communication and Adhesion 15:207-218 (2008). In vivo methods may also be used. See, for example, the methods of Danesh-Meyer, H V, et al. Connexin43 mimetic peptide reduces vascular leak and retinal ganglion cell death following retinal ischemia. Brain, 135:506-520 (2012); Davidson, J O, et al. (2012). Connexin hemichannel blockade improves outcomes in a model of fetal ischemia. Annals of Neurology 71:121-132 (2012).

One method for use in identifying or evaluating the ability of a compound to block hemichannels, comprises: (a) bringing together a test sample and a test system, said test sample comprising one or more test compounds, and said test system comprising a system for evaluating hemichannel block, said system being characterized in that it exhibits, for example, elevated transfer of a dye or labelled metabolite, for example, in response to the introduction of high glucose, hypoxia or ischemia to said system, a mediator of inflammation, or other compound or event that induces hemichannel opening, such as a drop in extracellular Ca²⁺; and, (b) determining the presence or amount of a rise in, for example, the dye or other labelled metabolite(s) in said system. Positive and/or negative controls may be used as well. Optionally, a predetermined amount of hemichannel blocker (e.g., Peptide5 or Xiflam) may be added to the test system.

### Dosage Forms and Formulations and Administration

All descriptions with respect to dosing, unless otherwise expressly stated, apply to the hemichannel blockers of the invention. The hemichannel blockers can be dosed, administered or formulated as described herein. In one embodiment, a composition comprising, consisting essentially of, or consisting of one or more hemichannel blockers are administered. Hemichannel blocker(s) may be administered QD, BID, TID, QID, or in weekly doses, e.g., QWK (once-per-week) or BIW (twice-per-week). They may also be administered monthly using doses described herein. They may also be administered PRN (i.e., as needed), and HS (hora somni, i.e., at bedtime). The hemichannel blockers can be administered to a subject in need of treatment. Thus, in accordance with the invention, there are provided formulations by which a connexin hemichannel, for example, a connexin 43 hemichannel or a connexin 45 hemichannel or a connexin 36 hemichannel can be modulated to decrease its open probability in a transient and site-specific manner. The hemichannel blockers may be present in the formulation in a substantially isolated form. It will be understood that the product may be mixed with carriers or diluents that will not interfere with the intended purpose of the product and still be regarded as substantially isolated. A product of the invention may also be in a substantially purified form, in which case it will generally comprise about 80%, 85%, or 90%, e.g. at least about 88%, at least about 90, 95 or 98%, or at least about 99% of a small molecule hemichannel blocker, for example, or dry mass of the preparation.

Administration of a hemichannel blocker to a subject may occur by any means capable of delivering the agents to a target site within the body of a subject. By way of example, a hemichannel blocker may be administered by one of the following routes: oral, topical, systemic (e.g., intravenous, intra-arterial, intra-peritoneal, transdermal, intranasal, or by suppository), parenteral (e.g. intramuscular, subcutaneous, or intravenous or intra-arterial injection), by implantation (including peritoneal, subcutaneous and ocular implantation), and by infusion through such devices as osmotic pumps, transdermal patches, and the like. Exemplary administration routes are also outlined in: Binghe, W. and B. Wang (2005). Drug delivery: principles and applications, Binghe Wang, Teruna Siahaan, Richard Soltero, Hoboken, N. J. Wiley-Interscience, c2005. In one embodiment, a hemichannel blocker is administered systemically. In another embodiment, a hemichannel blocker is administered orally. In another embodiment, a hemichannel blocker is administered topically onto or directly into the eye, for example.

In some aspects, the hemichannel blocker may be provided as, or in conjunction with, an implant. In some aspects, the implant may provide for slow-release, controlled-release or sustained-release delivery, with or without a burst dose. In some embodiments, a microneedle, needle, iontophoresis device or implant may be used for administration of the hemichannel blocker. The implant can be, for example, a dissolvable disk material such as that described in S. Pflugfelder et al., ACS Nano, 9 (2), pp 1749-1758 (2015). In some aspects, the hemichannel blockers, e.g. connexin 43 hemichannel blockers, of this invention may be administered via intraventricular, and/or intrathecal, and/or extradural, and/or subdural, and/or epidural routes.

The hemichannel blocker may be administered once, or more than once, or periodically. It may also be administered PRN (as needed) or on a predetermined schedule or both. In some aspects, the hemichannel blocker is administered daily, weekly, monthly, bi-monthly or quarterly, or in any combination of these time periods. For example, treatment may be administered daily for a period, follow by weekly and/or monthly, and so on. Other methods of administering blockers are featured herein. In one aspect, a hemichannel blocker is administered to a patient at times on or between days 1 to 5, 10, 30, 45, 60, 75, 90 or day 100 to 180, in amounts sufficient to treat the patient.

A hemichannel blocker, such as compounds of Formula I, for example Xiflam, and analogs or prodrugs of any of the foregoing compounds, or a compound of Formula II, may be administered alone or in combination with one or more additional ingredients and may be formulated into pharmaceutical compositions including one or more pharmaceutically acceptable excipients, diluents and/or carriers. In some embodiments, the hemichannel blocker, such as compounds of Formula I, for example Xiflam (tonabersat), and analogs or prodrugs of any of the foregoing compounds, or a compound of Formula II, may be orally administered in a composition comprising a foodstuff. In some embodiments, the foodstuff is peanut butter or a hazelnut-based cream. Without being bound by theory, it is believed that the relatively hydrophobic compounds of Formula I, including tonabersat, or Formula II, are slowly released after encapsulation in the emulsified fats of a foodstsuff (e.g., peanut butter), resulting in a prolonged therapeutic lifetime.

As used herein, the term “pharmaceutically acceptable diluents, carriers and/or excipients” is intended to include substances that are useful in preparing a pharmaceutical composition, may be co-administered with compounds of Formula I, for example Xiflam, and analogs of any of the foregoing compounds, or compounds of Formula II, while allowing it to perform its intended function, and are generally safe, non-toxic and neither biologically nor otherwise undesirable. Pharmaceutically acceptable diluents, carriers and/or excipients include those suitable for veterinary use as well as human pharmaceutical use. Suitable carriers and/or excipients will be readily appreciated by persons of ordinary skill in the art, having regard to the nature of compounds of Formula I, for example Xiflam, and analogs of any of the foregoing compounds. However, by way of example, diluents, carriers and/or excipients include solutions, solvents, dispersion media, delay agents, polymeric and lipidic agents, emulsions and the like. By way of further example, suitable liquid carriers, especially for injectable solutions, include water, aqueous saline solution, aqueous dextrose solution, and the like, with isotonic solutions being preferred for intravenous, intraspinal, and intracisternal administration and vehicles such as liposomes being also especially suitable for administration of agents.

Compositions may take the form of any standard known dosage form including tablets, pills, capsules, semisolids, powders, sustained release formulation, solutions, suspensions, elixirs, aerosols, liquids for injection, gels, creams, transdermal delivery devices (for example, a transdermal patch), inserts such as organ inserts, e.g., skin or eye, or any other appropriate compositions. Persons of ordinary skill in the art to which the invention relates will readily appreciate the most appropriate dosage form having regard to the nature of the condition to be treated and the active agent to be used without any undue experimentation. It should be appreciated that one or more of hemichannel blocker, such as compounds of Formula I, for example Xiflam, and analogs of any of the foregoing compounds, and/or a compound of Formula II, may be formulated into a single composition. In certain embodiments, preferred dosage forms include an injectable solution, an implant (preferably a slow-release, controlled-release or sustained-release implant, with or without a burst dose) and an oral formulation.

Compositions useful in the invention may contain any appropriate level of hemichannel blocker, such as compounds of Formula I, for example Xiflam, and analogs of any of the foregoing compounds, and/or a compound of Formula II, having regard to the dosage form and mode of administration. However, by way of example, compositions of use in the invention may contain from approximately 0.1% to approximately 99% by weight, preferably from approximately 1% to approximately 60% of a hemichannel blocker, depending on the method of administration.

In addition to standard diluents, carriers and/or excipients, a composition in accordance with the invention may be formulated with one or more additional constituents, or in such a manner, so as to enhance the activity or bioavailability of hemichannel blocker, such as compounds of Formula I, for example Xiflam, and analogs of any of the foregoing compounds, and/or a compound of Formula II, help protect the integrity or increase the half-life or shelf life thereof, enable slow release upon administration to a subject, or provide other desirable benefits, for example. For example, slow-release vehicles include macromers, poly(ethylene glycol), hyaluronic acid, poly(vinylpyrrolidone), or a hydrogel. By way of further example, the compositions may also include preserving agents, solubilizing agents, stabilizing agents, wetting agents, emulsifying agents, sweetening agents, coloring agents, flavoring agents, coating agents, buffers and the like. Those of skill in the art to which the invention relates can identify further additives that may be desirable for a particular purpose.

As noted, hemichannel blockers may be administered by a sustained-release system. Suitable examples of sustained-release compositions include semi-permeable polymer matrices in the form of shaped articles, e.g., films, or microcapsules. Sustained-release matrices include polylactides (U.S. Pat. No. 3,773,919; EP 58,481), copolymers of L-glutamic acid and gamma-ethyl-L-glutamate, poly(2-hydroxyethyl methacrylate), ethylene vinyl acetate, or poly-D-(−)-3-hydroxybutyric acid (EP 133,988). Sustained-release compositions also include a liposomally entrapped compound. Liposomes containing hemichannel blockers may be prepared by known methods, including, for example, those described in: DE 3,218,121; EP 52,322; EP 36,676; EP 88,046; EP 143,949; EP 142,641; Japanese Pat. Appln. 83-118008; U.S. Pat. Nos. 4,485,045 and 4,544,545; and EP 102,324. Ordinarily, the liposomes are of the small (from or about 200 to 800 Angstroms) unilamellar type in which the lipid content is greater than about 30 mole percent cholesterol, the selected proportion being adjusted for the most efficacious therapy. Slow release delivery using PGLA nano- or microparticles, or in situ ion activated gelling systems may also be used, for example.

Additionally, it is contemplated that a hemichannel blocker pharmaceutical composition for use in accordance with the invention may be formulated with additional active ingredients or agents which may be of therapeutic or other benefit to a subject in particular instances. Persons of ordinary skill in the art to which the invention relates will appreciate suitable additional active ingredients having regard to the description of the invention herein and nature of the EMT- and/or EndMT-related disorder to be treated.

Additionally, it is contemplated that a hemichannel blocker pharmaceutical composition for use in accordance with the invention may be formulated in a candy or food item, e.g., as a “gummy” pharmaceutical.

The compositions may be formulated in accordance with standard techniques as may be found in such standard references as Gennaro A R: Remington: The Science and Practice of Pharmacy, 20^{th} ed., Lippincott, Williams & Wilkins, 2000, for example. However, by way of further example, the information provided in US2013/0281524 or U.S. Pat. No. 5,948,811 may be used.

Any container suitable for storing and/or administering a pharmaceutical composition may be used for a hemichannel blocker product for use in a method of the invention.

The hemichannel blocker(s), for example, connexin 43 hemichannel blocker(s) may, in some aspects, be formulated to provide controlled and/or compartmentalized release to the site of administration. In some aspects of this invention, the formulations may be immediate, or extended or sustained release dosage forms. In some aspects, the dosage forms may comprise both an immediate release dosage form, in combination with an extended and/or sustained release dosage form. In some aspects both immediate and sustained and/or extended release of hemichannel blocker(s) can be obtained by combining hemichannel blocker(s) in an immediate release form. In some aspects of this invention the hemichannel blockers are, for example, connexin 43 blockers or other hemichannel blockers of this disclosure. In some aspects of this invention, the dosage forms may be implants, for example, biodegradable or nonbiodegradable implants.

The invention comprises methods for modulating the function of a hemichannel for the treatment and reversal or substantial reversal or amelioration of various disorders. Methods of the invention comprise administering a hemichannel blocker, alone or in a combination with one or more other agents or therapies as desired.

Administration of a hemichannel blocker, and optionally one or more other active agents, may occur at any time during the progression of a disorder, or prior to or after the development of a disorder or one or more symptom of a disorder. In one embodiment, a hemichannel blocker is administered periodically for an extended period to assist with ongoing management or reversal of symptoms. In another embodiment, a hemichannel blocker is administered periodically for an extended period or life-long to prevent or delay the development of or eliminate an EMT- and/or EndMT-related disorder.

In some embodiments, the hemichannel blockers, for example, a connexin 43 hemichannel blocker (e.g., compounds of Formula (I), including tonabersat, or compounds of Formula (II)), can be administered as a pharmaceutical composition comprising one or a plurality of particles. In some aspects, the pharmaceutical composition may be, for example, an immediate release formulation or a controlled release formulation, for example, a delayed release particle. In other aspects, hemichannel blockers can be formulated in a particulate formulation one or a plurality of particles for selective delivery to a region to be treated. In some embodiments, the particle can be, for example, a nanoparticle, a nanosphere, a nanocapsule, a liposome, a polymeric micelle, or a dendrimer. In some embodiments, the particle can be a microparticle. The nanoparticle or microparticle can comprise a biodegradable polymer. In other embodiments, the hemichannel blocker is prepared or administered as an implant, or matrix, or is formulated to provide compartmentalized release to the site of administration. In some embodiments, the pharmaceutical composition of the hemichannel blockers, for example, a connexin 43 hemichannel blocker (e.g., compounds of Formula (I), including tonabersat, or compounds of Formula (II)) does not comprise microparticles.

In some embodiments, as noted, the formulated hemichannel blocker is a connexin 37 or connexin 40 or connexin 43 or connexin 45 hemichannel blocker, by way of example. Connexin 36 or connexin 37 or connexin 40 or connexin 43 or connexin 45 blockers are preferred. Most preferred are connexin 36 and connexin 43 hemichannel blockers. Especially preferred are connexin 43 hemichannel blockers. As used herein, “matrix” includes for example, matrices such as polymeric matrices, biodegradable or non-biodegradable matrices, and other carriers useful for making implants or applied structures for delivering the hemichannel blockers. Implants include reservoir implants and biodegradeable matrix implants.

### Articles of Manufacture/Kits of Combinations of Connexin Hemichannel Blockers

In another embodiment of the invention, an article of manufacture, or “kit”, containing materials useful for treating the EMT- and/or EndMT-related disease, disorder or condition described or referenced herein is provided. The kit comprises a container comprising, consisting essentially of, or consisting of connexin hemichannel blocker/inhibitor. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, e.g., bottles, vials, syringes, blister pack, etc. The container may be formed from a variety of materials such as glass or plastic. The container holds a hemichannel blocker, or a formulation thereof, which is effective for treating the condition and may have a sterile access port (e.g., the container may be an intravenous solution bag or a vial having a stopper pierceable by a hypodermic injection needle). The label or package insert indicates that the composition is used for treating the condition of choice, such any EMT- and/or EndMT-related disease, disorder and/or condition, including those described or referenced herein. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the hemichannel blocker to a patient in need thereof, or provide instruction to access the directions online or in the cloud.

Articles of manufacturer are also provided, comprising, consisting essentially of, or consisting of a vessel containing a hemichannel blocker compound, composition or formulation and instructions for use for the treatment of a subject. For example, in another aspect, the invention includes an article of manufacture comprising, consisting essentially of, or consisting of a vessel containing a therapeutically effective amount of one or more connexin hemichannel blockers, including small molecules, together with instructions for use, including use for the treatment of a subject.

In some aspects, the article of manufacture may comprise a matrix that comprises one or more connexin hemichannel blockers, such as a small molecule hemichannel blocker, alone or in combination.

### Doses, Amounts and Concentrations

As will be appreciated, the dose of hemichannel blocker administered, the period of administration, and the general administration regime may differ between subjects depending on such variables as the target site to which it is to be delivered, the severity of any symptoms of a subject to be treated, the type of disorder to be treated, size of unit dosage, the mode of administration chosen, and the age, sex and/or general health of a subject and other factors known to those of ordinary skill in the art.

Included herein are methods for treating an EMT- and/or EndMT-related disease, disorder or condition in a subject, comprising, e.g., administering to said subject an effective amount of a hemichannel blocker, including, for example, N-[(3S,4S)-6-acetyl-3-hydroxy-2,2-dimethyl-3,4-dihydrochromen-4-yl]-3-chloro-4-fluorobenzamide (Xiflam). In some embodiments, the doses are as described herein. survival-promoting amount is about 10 to about 200 mg per day, or in some embodiments, from about 3.5 to 350 mg per day. In other embodiments, the survival-promoting amount is about 20 to about 100 mg per day. These amounts may be administered in single or divided doses, e.g., BID. Preferred are doses ranging from about 1.0 to about 10 mg/kg per day. Doses may be, for example, about 1.0, 1.1., 1.2, 1.3, 1.4, 1.5 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4., 4.5, 4.6, 4.7, 4.8, 4.9, etc., or about 10.0 mg/kg per day, or any range between any two of the recited doses.

An especially preferred daily dose is about 1-3 mg/kg per dose or per day, the latter being in single or divided doses (e.g., BID). Thus, for example, with a subject weighing about 70 kg, 90 kg, or 100 kg, the amount administered would be about 70, 140 or 210 mg per day or per dose, about 90, 180 or 270 mg per day or per dose, or about 100, 200 or 300 mg per day or per dose, respectively. With tonabersat, for example, these doses will provide an effective, peak steady state concentration of a hemichannel blocker after about 10 days.

In some embodiments, the hemichannel inhibitor is administer once per week (QWK). In one QWK dosing embodiment, the hemichannel blocker compound is administered in a slow-release, sustained-release or controlled release oral or implant formulation, with or without a 10-20% burst dose, or other desired burst dose. Implant formulations, for example, ocular implant formulations, preferably range from disposed in a slow-release, sustained-release or controlled release oral or implant formulation.

### Manufacture and Purity

Small molecule hemichannel blockers, including those of Formula I and II may be prepared as previously described.

In some embodiments, the formulations of this invention are substantially pure. By substantially pure is meant that the formulations comprise less than about 10%, 5%, or 1%, and preferably less than about 0.1%, of any impurity. In some embodiments the total impurities, including metabolites of the connexin 43 modulating agent, will be not more than 1-15%. In some embodiments the total impurities, including metabolites of the connexin 43 modulating agent, will be not more than 2-12%. In some embodiments the total impurities, including metabolites of the connexin 43 modulating agent, will be not more than 3-11%. In other embodiments the total impurities, including metabolites of the connexin 43 modulating agent, will be not more than 4-10%.

### EXAMPLES

The work described in these Examples evaluated and demonstrated the ability of modulation of connexin hemichannels with hemichannel blocker doses and dose regimens to attenuate epithelial-mesenchymal transition. Epithelial cells were either insulted with high glucose plus cytokines (HG+Cyt) alone, or treated with a connexin 43 hemichannel modulator according to Formula I (tonabersat) alongside the HG+Cyt insult, from passage 15 onwards. Markers of both epithelial and mesenchymal phenotypes were monitored along with cell migration rates and barrier function. The experiments show that hemichannel modulation plays a role in regulating epithelial-mesenchymal transition, and that hemichannel blockers can favorably modulate it.

### Example 1

### Methods

### Cell Culture

Human adult retinal pigment epithelial cells (ARPE-19; American Type Culture Collection, USA) were cultured in Dulbecco's modified Eagle medium F-12 (DMEM-F12; Thermofisher Scientific Inc., USA) supplemented with 10% foetal bovine serum (PBS: Invitrogen, USA) and a 1× antibiotics and antimycotics mixture (AA, 100× stock) at 37° C. in a humidified 5% CO₂ incubator. Cells were grown in T75 flasks, and the medium was changed twice per week until confluent.

### High Glucose (HG) and Cytokine Challenge

At passages 15-18, cells were plated at 2.5×10⁵ cells/mL in 8-well chamber slides for immunohistochemical studies, 6-well plates with inserts for fluorescein isothiocyanate (FITC)-dextran dye leak and transepithelial electrical resistance (TEER) measurements, and 24-well plates for cell migration studies. Once confluent, cells were treated as three separate groups: untreated, insult of HG+Cyt, or HG+Cyt insult plus tonabersat treatment (HG+Cyt+Ton). The insult of HG+Cyt was used to induce DR-like conditions as has been previously described (Kuo et al., 2020; Mugisho et al., 2018a, 2018b), and consisted of a combination of 32.5 mM HG and the pro-inflammatory cytokines; tumour necrosis factor alpha (TNF-α; 10 ng/mL; Peprotech, USA) and interleukin-1 beta (IL-1β; 10 ng/mL; Peprotech, USA).

### Application of Treatments

Tonabersat (MedChemExpress, NJ, USA) was administered at a concentration of 100 μM to cells at the same time as the HG+Cyt insult (HG+Cyt+Ton). To achieve this tonabersat was dissolved in 100% DMSO at a concentration of 100 mM and then 1 μl of the stock solution was added to 999 μl of culture medium containing HG+Cyt. Cells were incubated under treatment conditions for 72 h unless otherwise stated. Brightfield images were taken using a light microscope at 24, 48, and 72 h post-treatment. All experiments were repeated thrice.

### Immunocytochemistry

Cells were fixed with 4% paraformaldehyde for 10 min and permeabilized with 0.1% Triton X-100 in phosphate-buffered saline (PBS) for 10 min. Cells were blocked in normal goat or horse serum for 1 h, and then incubated overnight at 4° C. with either mouse anti-RPE65 (1:1000; Abcam, UK) and goat anti-α-SMA (1:200; Abcam, UK), or rabbit anti-ZO-1 (1:1000; Invitrogen, USA). Two 10 min washes followed in PBS, after which cells were incubated at room temperature for 2 h with their respective secondary antibodies; donkey anti-rabbit Alexa-488 (1:500; Abcam, UK), donkey anti-mouse Alexa-488 (1:500; Abcam, UK) or donkey anti-goat Cy3 (1:500; Invitrogen, USA). Cells were washed in PBS twice for 10 min. Cell nuclei were stained with DAPI (1:1000; Sigma-Aldrich, USA), and slides were mounted using Citifluor™ anti-fade reagent and sealed with nail varnish.

### Image Analysis and Protein Quantification

Fluorescence images were taken on an Olympus FV1000 confocal laser scanning microscope (Olympus, Japan), and processed using Olympus FV10-ASW viewer and ImageJ software (Version 1.52a, National Institute of Health, USA). Five images were taken from a single chamber per condition and repeated in three separate experiments. RPE65 and α-SMA expression was quantified by measuring the mean fluorescence intensity (MFI). Data is presented relative to the respective untreated group. The ratio of α-SMA to RPE65 expression was additionally calculated by (α-SMA MFI/RPE65 MFI). ZO-1 was qualitatively assessed for changes in localisation.

### Cell Migration Assay

To determine cell migration rates following 72 h treatment, a scrape wound was created in the cell monolayer by drawing a 1000 μL pipette tip vertically over the cells by hand, and the width of the wound measured over time. Treatment effects were observed in duplicate wells, with cells incubated in the various treatment media (untreated, HG+Cyt, HG+Cyt+Ton) throughout the cell migration study. Images were taken of cells pre-scrape, immediately post-scrape, and at 4 and 24 h post-scrape using a light microscope. Five images were taken per well, with the same sections of the scrape imaged each time. The width of the scrape was measured with the line tool and “measure” feature in ImageJ at eight regular intervals (guided using a grid overlay) within each image. Mean values for each of the five images were then used for statistical analysis. Duplicate wells were used, creating a sample size of ten per condition. The scrape wound width was quantified relative to the post-scrape width at time 0 h and converted to a percentage scrape closure for each condition. Scrape wound percentage closure was compared between treatment groups within given timepoints, and across time within treatment groups.

### Measurement of FITC-Dextran Paracellular Permeability

The movement of a 70,000 Da FITC-dextran (D1820, Thermofisher Scientific Inc., USA) across a monolayer of cells was evaluated as previously described (Kuo et al., 2020). Briefly, following treatment, 1000 μL of spent medium in each plate insert was replaced by 1000 μL of FITC-dextran in media (10 μg/mL) and incubated for 15 min Inserts were removed, and media samples from the well base were transferred to 96-well plates for quantification by spectrophotometry (excitation 490 nm and emission 520 nm). FITC-dextran permeability was expressed relative to untreated wells which contained cells with no treatment. There were three wells per condition, each sampled 10 times.

### Measurement of Transepithelial Electrical Resistance (TEER)

To determine the effect of conditions on the TEER of the cell monolayer, cells were again seeded into the inserts of 6-well Transwell® plates (Corning Incorporated, USA), with additional medium in the base. Once confluent, the medium in the inserts was replaced with the three conditions (untreated media, HG+Cyt or HG+Cyt+Ton) for 72 h. TEER measurements were then obtained at 0, 24, 48 and 72 h following treatment addition, using the EVOM2 (World Precision Instruments, USA) with an STX3 electrode. TEER values were quantified relative to the 0 h values for each respective group. There were three wells per condition, each sampled six times.

### Statistical Analysis

Data are presented as arithmetic mean+S.E.M. on bar graphs and mean±S.E.M. on line graphs. In the case of RPE65 and α-SMA expression, and FITC-dextran permeability, data was quantified relative to the respective untreated group. Statistical comparisons were performed using one- or two-way ANOVA with post-hoc tests. The specific statistical method used for each data set is presented in the respective figure legend. GraphPad Prism version 8.2.1 for Windows (GraphPad Software, San Diego, Calif. USA) was used for all statistical analysis. Adjusted p≤0.05 was considered to indicate a statistically significant difference.

### Example 2

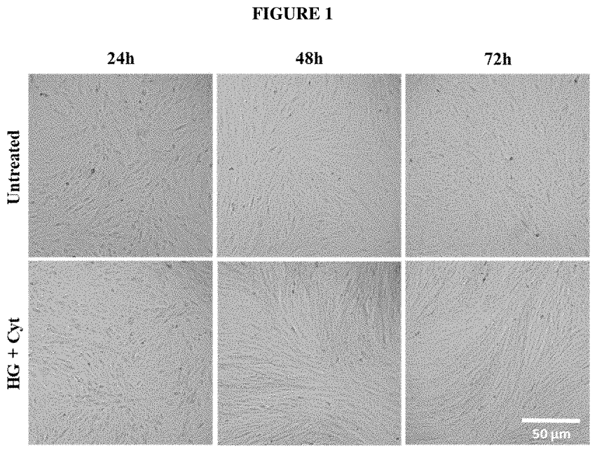

This Example shows that co-application of HG+Cyt led a change in cell morphology. Normal ARPE-19 cells in culture have a truncated fibroblastic form, in places appearing almost cuboidal. HG+Cyt application induced differentiation into an elongated and stretched phenotype starting within 24 h and becoming very apparent within 48 h (FIG. 1). The effect of HG+Cyt treatment was further intensified with longer treatment periods, with cells insulted over a 72 h period displaying both greater elongation and a higher proportion of elongated cells compared with HG+Cyt treated cells after 24 h incubation.

### Example 3

This Example shows that epithelium specific phenotypic marker RPE65 were down-regulated following HG+Cyt insult, but maintained in the presence of a hemichannel inhibitor (tonabersat). Expression levels of RPE65, a cellular marker specific to RPE cells, was analyzed using quantitative immunocytochemistry in order to determine changes in epithelial cell phenotype.

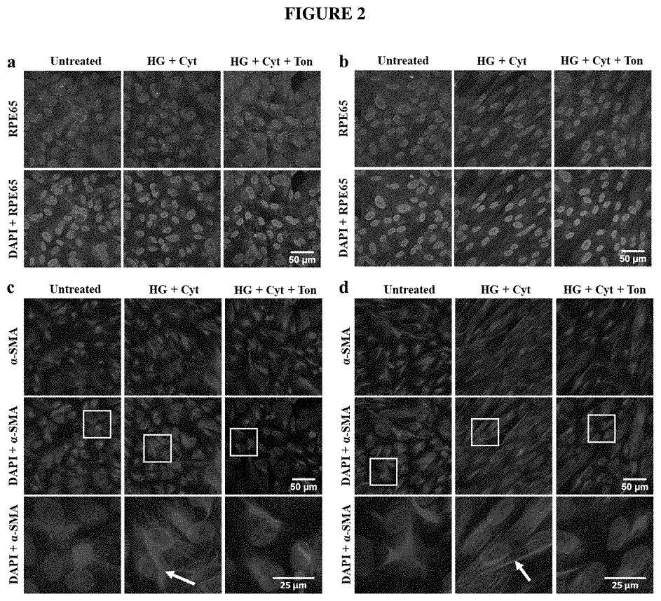

RPE65 expression was significantly influenced by treatment conditions after both 24 h (F (2,11)=4.041, p=0.0483) and 72 h (F (2, 10)=8.504, p=0.0070) (FIG. 2). Results showed that the HG+Cyt+Ton group (112.6±6.4%) exhibited significantly higher RPE65 expression at 24 h than the insulted HG+Cyt group (84.5±6.0%, p=0.0286 (FIG. 2b).

By 72 h, RPE65 expression was significantly lower in the HG+Cyt group (81.2±2.8% compared to untreated levels at 72 h) than the untreated group (100.0±4.5%, p=0.0040) (FIG. 2d). The HG+Cyt+Ton group now fell in-between, not being significantly different to either group.

### Example 4

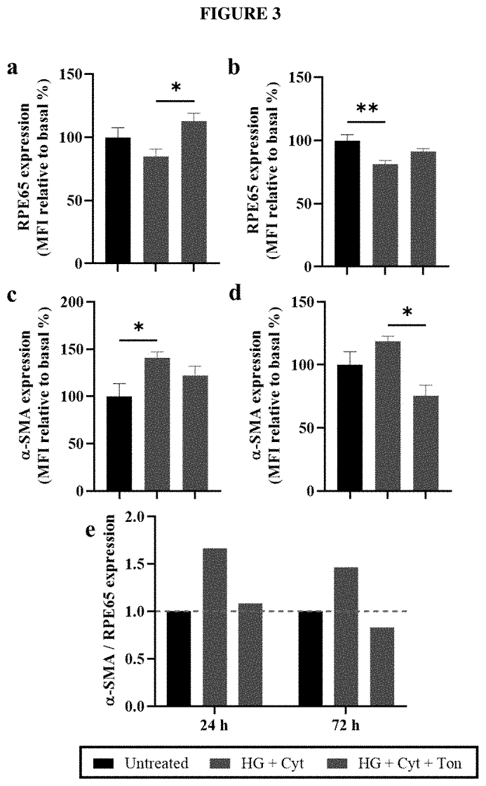

This Example showed that HG+Cyt conditions led to increased expression of α-SMA, a marker for mesenchymal phenotype differentiation, but was prevented by treatment with a hemichannel inhibitor (tonabersat). A significant effect of treatment conditions on the mesenchymal marker α-SMA was determined by one-way ANOVA at both 24 h (F (2,11)=3.358, p=0.072) and 72 h (F (2, 10)=5.889, p=0.0204) (FIG. 3). Dunnett's multiple comparison test showed that after 24 h, α-SMA expression was significantly increased following HG+Cyt (140.6±6.4%) relative to untreated (100.0±13.7%, p=0.0459) conditions (FIG. 3c). At 72 h, HG+Cyt+Ton (75.5±8.4% compared to untreated levels at 72 h) had significantly lower α-SMA expression than the HG+Cyt group (118.8±4.0%, p=0.0119) (FIG. 3d). Additionally, the location of α-SMA within cells was seen to vary depending on treatment conditions (FIG. 3a,c), with expression delineating a more fibrous nature after insult and with increasing incubation time, than untreated or HG+Cyt+Ton treatment groups.

### Example 5

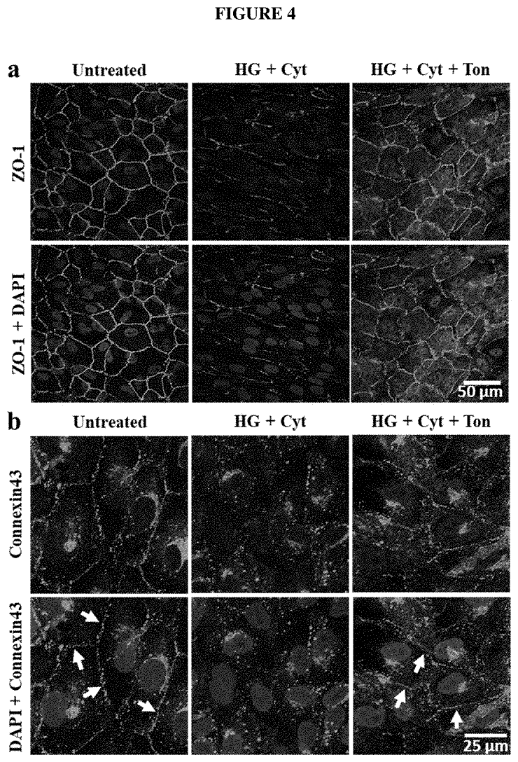

This Example showed that connexin43 hemichannel block prevents HG+Cyt induced increase in α-SMA to RPE65 expression ratio. In combining the changes seen in both α-SMA and RPE65 expression, it was demonstrated that HG+Cyt insult results in an increase in the α-SMA to RPE65 expression ratio compared to untreated conditions at both 24 and 72 h (24 h: untreated=1.0, HG+Cyt=1.7; 72 h: untreated=1.0, HG+Cyt=1.5) (FIG. 4).

Further, addition of the hemichannel inhibitor, tonabersat, (HG+Cyt+Ton) prevented this increase, maintaining the ratio at around 1, as in the untreated group (24 h=1.1; 72 h=0.8).

### Example 6

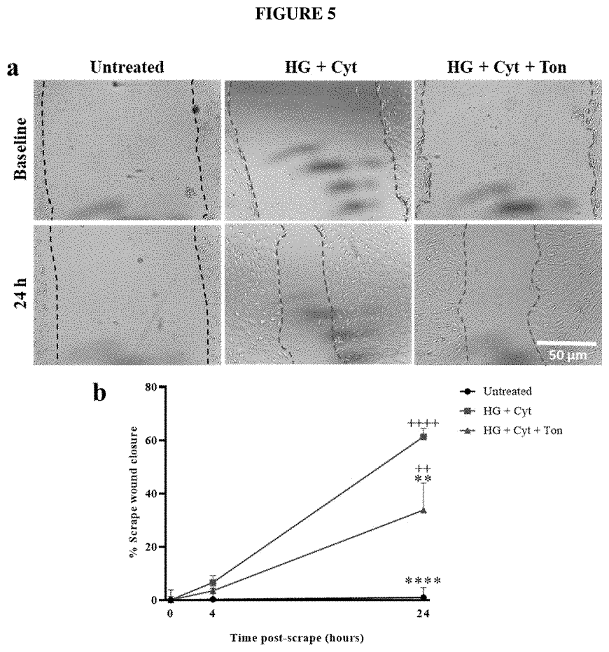

This Example showed that connexin43 hemichannel block with a compound according to Formula I (tonabersat) reduced HG+Cyt induced cell migration. Scrapes in all conditions began with a similar width (p>0.9999) (FIG. 5). At 4 h post-scraping, no significant treatment effect on wound closure was observed (F (2, 27)=1.148, p=0.3322) and there was no significant difference in percentage closure compared to their respective 0 h scrape widths for any of the treatment conditions (untreated, p=0.9846; HG+Cyt, p=0.1675; HG+Cyt+Ton, p=0.8752). However by 24 h, treatment conditions were found to affect scape closure (F (2, 27)=23.76, p<0.0001) (FIG. 5b). HG+Cyt led to a significant reduction in scrape width (61.3±3.1%, p<0.0001) compared with 0 h, with HG+Cyt+Ton treatment (33.8±9.6%) also resulting in significant scrape closure between 0 and 24 h (p=0.0015), although to a lesser extent. The untreated group saw no significant change in scrape width across time (p=0.9846). Comparison of scrape width at 24 h between treatment conditions demonstrated that HG+Cyt (61.3±3.1%) accelerated scrape closure compared with the untreated group (0.9±3.7%, p≤0.0001). HG+Cyt+Ton treatment resulted in a reduced scrape wound closure compared with the HG+Cyt insult alone (33.8±9.6%, p=0.0028).

### Example 7

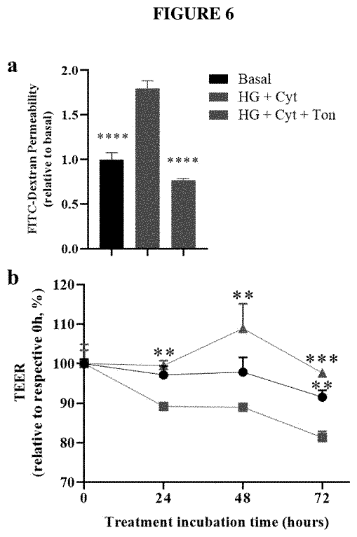

This Example showed that tight junction integrity was compromised by HG+Cyt insult, but maintained by co-application of the hemichannel blocker, tonabersat. ZO-1 is a tight junction protein, normally located on the cytoplasmic membrane of cells. After immunocytochemical labelling for ZO-1, cells in untreated conditions showed clear localisation of ZO-1 at the cell membranes with little cytoplasmic labelling (FIG. 6a). In cells exposed to HG+Cyt, however, a loss of ZO-1 membrane cell-cell interface localization was seen. HG+Cyt+Ton treatment maintained ZO-1 localization at the cell membrane. The ZO-1 cell membrane labelling was slightly less distinct compared to the untreated group but nonetheless essentially normal whilst in the HG+Cyt insulted cells limited ZO-1 membrane localization remained.

### Example 8

To determine the effect of treatment conditions on paracellular permeability, the passage of a large FITC-dextran molecule across the cell monolayer was studied. A significant effect of treatment on paracellular permeability was observed (F (2, 27)=58.72, p≤0.0001), with significantly higher FITC-dextran permeability following HG+Cyt insult (1.79±0.09) than both untreated (1.00±0.08, p≤0.0001) and HG+Cyt+Ton (0.77±0.02, p≤0.0001) groups (FIG. 6b). This Example showed that HG+Cyt induced an increase in paracellular permeability (dye passage) which was prevented by treatment with a hemichannel inhibitor (tonabersat).

### Example 9

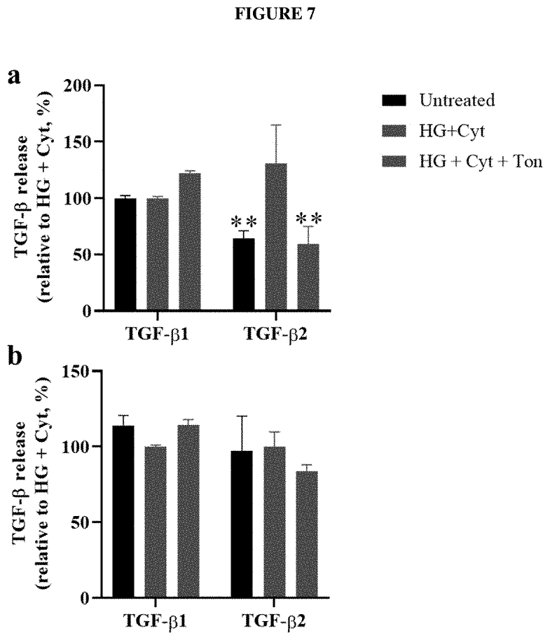

This Example showed that trans-epithelial electrical resistance was compromised by the HG+Cyt insult, but was maintained by co-application of the hemichannel inhibitor, tonabersat. Cells in all conditions started with the same TEER (p=0.7625). However, within 24 h a significant treatment effect on TEER was observed (p=0.0024) (FIG. 7). At 24 h, the HG+Cyt group (89.2±1.0%) had significantly lower TEER than both untreated (94.9±0.4%, p=0.0292) and HG+Cyt+Ton (99.5±1.2%, p=0.0015) groups. HG+Cyt+Ton at 48 h again maintained a higher TEER (114.9±3.2%, p=0.0029) than HG+Cyt alone (89.0±0.8%), while at 72 h TEER was again significantly higher for untreated (91.5±1.7%, p=0.0048) and HG+Cyt+Ton (97.7±1.1%, p=0.0004) conditions compared with HG+Cyt alone (81.3±1.6%).

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognize that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, and in embodiments or examples of the present invention, any of the terms “comprising”, “consisting essentially of”, and “consisting of” may be replaced with either of the other two terms in the specification. Thus, for example, a composition “comprising” certain listed ingredients also provides express written description support for and may also be claimed as a composition “consisting essentially of” or “consisting of” the listed ingredients. Similarly, a method “comprising” certain steps also provides express written description support for and may also be claimed as a composition “consisting essentially of” or “consisting of” the listed steps.

The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

### CITATIONS

Campochiaro, P. A., Aiello, L. P. & Rosenfeld, P. J. (2016). Anti-vascular endothelial growth factor agents in the treatment of retinal disease: from bench to bedside. Ophthalmology. [Online]. 123 (10). p. pp. S78-S88. Available from: http://dx.doi.org/10.1016/j.ophtha.2016.04.056.
Che, D., Zhou, T., Lan, Y., Xie, J., Gong, H., Li, C., Feng, J., Hong, H., Qi, W., Ma, C., Wu, Q., Yang, X. & Gao, G. (2016). High glucose-induced epithelial-mesenchymal transition contributes to the upregulation of fibrogenic factors in retinal pigment epithelial cells. International Journal of Molecular Medicine. 38 (6). p. pp. 1815-1822.
Chen, X., Ye, S., Xiao, W., Luo, L. & Liu, Y. (2014). Differentially expressed microRNAs in TGFβ2-induced epithelial-mesenchymal transition in retinal pigment epithelium cells. International Journal of Molecular Medicine. 33 (5). p. pp. 1195-1200.
Cunha-Vaz, J. (2009). The blood-retinal barrier in retinal disease. European Ophthalmic Review. 3. p. pp. 105-108.
Cunha-Vaz, J. G. (2004). The blood-retinal barriers system. Basic concepts and clinical evaluation. Experimental Eye Research. 78 (3). p. pp. 715-721.
Dhoot, D. S. & Avery, R. L. (2016). Vascular endothelial growth factor inhibitors for diabetic retinopathy. Current Diabetes Reports. [Online]. 16 (122). p. pp. 1-7. Available from: http://dx.doi.org/10.1007/s11892-016-0825-4.
Hyttinen, J. M. T., Kannan, R., Felszeghy, S., Niittykoski, M., Salminen, A. & Kaarniranta, K. (2019). The regulation of NFE2L2 (NRF2) signalling and epithelial-to-mesenchymal transition in age-related macular degeneration pathology. International Journal of Molecular Sciences. 20 (5800). p. pp. 1-35.
Jing, R., Qi, T., Wen, C., Yue, J., Wang, G., Pei, C. & Ma, B. (2019). Interleukin-2 induces extracellular matrix synthesis and TGF-β2 expression in retinal pigment epithelial cells. Development Growth and Differentiation. 61. p. pp. 410-418.
Kalluri, R. & Weinberg, R. A. (2009). The basics of epithelial-mesenchymal transition. The Journal of Clinical Investigation. 119 (6). p. pp. 1420-1428.
Klaassen, I., Van Noorden, C. J. F. & Schlingemann, R. O. (2013). Molecular basis of the inner blood-retinal barrier and its breakdown in diabetic macular edema and other pathological conditions. Progress in Retinal and Eye Research. [Online]. 34. p. pp. 19-48. Available from: http://dx.doi.org/10.1016/j.preteyeres.2013.02.001.
Kobayashi, M., Tokuda, K., Kobayashi, Y., Yamashiro, C., Uchi, S. H., Hatano, M. & Kimura, K. (2019). Suppression of epithelial-mesenchymal transition in retinal pigment epithelial cells by an MRTF-A inhibitor. Investigative Ophthalmology and Visual Science. 60 (2). p. pp. 528-537.
Kovach, J. L., Schwartz, S. G., Flynn, H. W. & Scott, I. U. (2012). Anti-VEGF treatment strategies for wet AMD. Journal of Ophthalmology. 2012 (786870). p. pp. 1-7.
Kroll, P., Michele Rodrigues, E. & Hoerle, S. (2007). Pathogenesis and classification of proliferative diabetic vitreoretinopathy. Ophthalmologica. 221 (2). p. pp. 78-94.
Kuo, C., Green, C. R., Rupenthal, I. D. & Mugisho, O. O. (2020). Connexin43 hemichannel block protects against retinal pigment epithelial cell barrier breakdown. Acta Diabetologica. [Online]. 57 (1). p. pp. 13-22. Available from: https://doi.org/10.1007/s00592-019-01352-3.
Lee, H., Hwang-Bo, H., Ji, S. Y., Kim, M. Y., Kim, S. Y., Park, C., Hong, S. H., Kim, G. Y., Song, K. S., Hyun, J. W. & Choi, Y. H. (2020). Diesel particulate matter 2.5 promotes epithelial-mesenchymal transition of human retinal pigment epithelial cells via generation of reactive oxygen species. Environmental Pollution. 262 (114301). p. pp. 1-10.
Li, X., Yan, X., Wang, Y., Wang, J., Zhou, F., Wang, H., Xie, W. & Kong, H. (2018). NLRP3 inflammasome inhibition attenuates silica-induced epithelial to mesenchymal transition (EMT) in human bronchial epithelial cells. Experimental Cell Research. [Online]. 362 (2). p. pp. 489-497. Available from: https://doi.org/10.1016/j.yexcr.2017.12.013.
Li, X., Zhao, M. & He, S. (2020). RPE epithelial-mesenchymal transition plays a critical role in the pathogenesis of proliferative vitreoretinopathy. Annals of Translational Medicine. 8 (6). p. pp. 263-263.
Lyon, H., Shome, A., Rupenthal, I. D., Green, C. R., & Mugisho, O. O. (2021). Tonabersat Inhibits Connexin43 Hemichannel Opening and Inflammasome Activation in an In Vitro Retinal Epithelial Cell Model of Diabetic Retinopathy. International Journal of Molecular Sciences, 22(1), 298. ARTN 298
Mony, S., Lee, S. J., Harper, J. F., Barwe, S. P. & Langhans, S. A. (2013). Regulation of Na,K-ATPase β1-subunit in TGF-β2-mediated epithelial-to-mesenchymal transition in human retinal pigmented epithelial cells. Experimental Eye Research. 115. p. pp. 113-122.
Mugisho, O. O., Green, C. R., Kho, D. T., Zhang, J., Graham, E. S., Acosta, M. L. & Rupenthal, I. D. (2018a). The inflammasome pathway is amplified and perpetuated in an autocrine manner through connexin43 hemichannel mediated ATP release. BBA—General Subjects. 1862. p. pp. 385-393.
Mugisho, O. O., Green, C. R., Squirrell, D. M., Bould, S., Danesh-Meyer, H. V., Zhang, J., Acosta, M. L. & Rupenthal, I. D. (2019). Connexin43 hemichannel block protects against the development of diabetic retinopathy signs in a mouse model of the disease. Journal of Molecular Medicine. 97 (2). p. pp. 215-229.
Mugisho, O. O., Rupenthal, I. D., Squirrell, D. M., Bould, S. J., Danesh-Meyer, H. V., Zhang, J., Green, C. R. & Acosta, M. L. (2018b). Intravitreal pro-inflammatory cytokines in non-obese diabetic mice: Modelling signs of diabetic retinopathy. PLoS ONE. 13 (8). p. pp. 1-20.
Shukal, D., Bhadresha, K., Shastri, B., Mehta, D., Vasavada, A., & Johar SR, K. (2020). Dichloroacetate prevents TGFβ-induced epithelial-mesenchymal transition of retinal pigment epithelial cells. Experimental Eye Research, 108072.
Song, S., Qiu, D., Luo, F., Wei, J., Wu, M., Wu, H., Du, C., Du, Y., Ren, Y., Chen, N., Duan, H. & Shi, Y. (2018). Knockdown of NLRP3 alleviates high glucose or TGFB1-induced EMT in human renal tubular cells. Journal of Molecular Endocrinology. 61 (3). p. pp. 101-113.
Tamiya, S. & Kaplan, H. J. (2016). Role of epithelial-mesenchymal transition in proliferative vitreoretinopathy. Experimental Eye Research. 142. p. pp. 26-31.
Tamiya, S., Liu, L. H. & Kaplan, H. J. (2010). Epithelial-mesenchymal transition and proliferation of retinal pigment epithelial cells initiated upon loss of cell-cell contact. Investigative Ophthalmology and Visual Science. 51 (5). p. pp. 2755-2763.
Wang, H. F., Ma, J. X., Shang, Q. L., An, J. Bin & Chen, H. T. (2017). Crocetin inhibits the proliferation, migration and TGF-β2-induced epithelial-mesenchymal transition of retinal pigment epithelial cells. European Journal of Pharmacology. 815. p. pp. 391-398.
Yang, I. H., Lee, J. J., Wu, P. C., Kuo, H. K., Kuo, Y. H. & Huang, H. M. (2020). Oxidative stress enhanced the transforming growth factor-β2-induced epithelial-mesenchymal transition through chemokine ligand 1 on ARPE-19 cell. Scientific Reports. [Online]. 10 (4000). p. pp. 1-10. Available from: http://dx.doi.org/10.1038/s41598-020-60785-x.

We claim:

1. A method for inhibiting epithelial-mesenchymal transition activity in a subject having a disease, disorder or condition characterized in part by pathological or unwanted epithelial-mesenchymal transition activity, comprising administering a hemichannel inhibitor to said subject in an amount effective to inhibit epithelial-mesenchymal transition activity.

2. The method of claim 1, wherein the hemichannel inhibitor is a connexin 43 hemichannel inhibitor.

3. The method of claim 1, wherein the hemichannel inhibitor is a small molecule hemichannel inhibitor.

4. The method of claim 1, wherein the hemichannel inhibitor is N-[(3S,4S)-6-acetyl-3-hydroxy-2,2-dimethyl-3,4-dihydrochromen-4-yl]-3-chloro-4-fluorobenzamide (tonabersat).

5. The method of claim 1, wherein the hemichannel inhibitor is a compound of Formula (I): 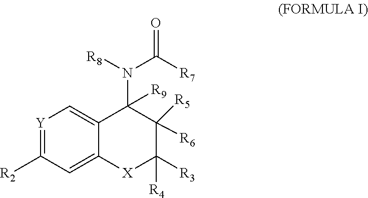
wherein Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—,
—CO—, —CH₂—, CH(OH), SO₂, SO, CH₂—O, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆alkyl or by C₂₋₇ alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C₁₋₆ alkyl amino, amino-C₁₋₆ alkyl, hydroxy-C₁₋₆ alkyl, halo-C₁₋₆ alkyl, C₁₋₆ acyloxy-C₁₋₆ alkyl, C₁₋₆alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆alkyl.

6. The method of claim 1, wherein the small molecule hemichannel blocker is a compound of Formula (II): 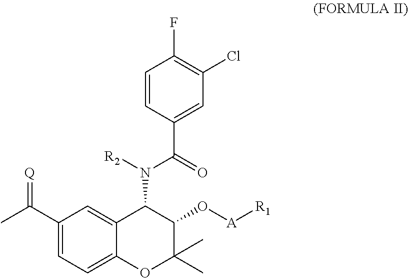
wherein
Q is O or an oxime of formula ═NHOR₄₃, wherein R₄₃ is
(i) selected from H, C₁₋₄ fluoroalkyl or optionally substituted C₁₋₄ alkyl, or
(ii) -A₃₀₀-R₃₀₀ wherein
A₃₀₀ is a direct bond, —C(O)O*—, —C(R₃)(R₄)O*—, —C(O)O—C(R₃)(R₄)O*—, or —C(R₃)(R₄)OC(O)O*— wherein the atom marked* is directly connected to R₃₀₀,
R₃ and R₄ are selected independently from H, fluoro, C₁₋₄ alkyl, or C₁₋₄ fluoroalkyl, or
R₃ and R₄ together with the atom to which they are attached form a cyclopropyl group,
R₃₀₀ is selected from groups [1], [2], [2A], [3], [4], [5] or [6];
R₂ is H or B—R₂₁,
A is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₁, R₃ and R₄ are selected independently from H, fluoro, C₁₋₄ alkyl, or C₁₋₄ fluoroalkyl, or R₃ and R₄ together with the atom to which they are attached form a cyclopropyl group,
R₁ is selected from groups [1], [2], [2A], [3], [4], [5] and [6] wherein the atom marked ** is directly connected to A: 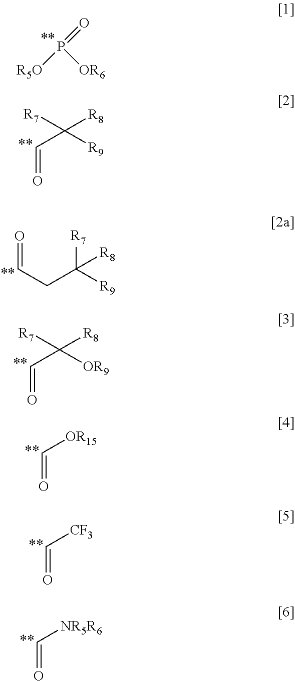
R₅ and R₆ are each independently selected from H, C₁₋₄ alkyl, C₁₋₄ fluoroalkyl, and benzyl;
R₇ is independently selected from H, C₁₋₄ alkyl, and C₁₋₄ fluoroalkyl;
R₈ is selected from:
(i) H, C₁₋₄ alkyl, or C₁₋₄ fluoroalkyl, or
(ii) the side chain of a natural or unnatural alpha-amino acid, or a peptide as described herein, or
(iii) biotin or chemically linked to biotin;
R₉ is selected from H, —N(R₁₁)(R₁₂), or —N⁺(R₁₁)(R₁₂)(R₁₃)X⁻, or —N(R₁₁)C(O)R₁₄ wherein R₁₁, R₁₂, and R₁₃ are independently selected from H, C₁₋₄ alkyl, or C₁₋₄ fluoroalkyl,
R₁₄ is H, C₁₋₄ alkyl, or C₁₋₄ fluoroalkyl,
R₁₅ is independently selected from C₁₋₄ alkyl and C₁₋₄ fluoroalkyl, and
X⁻ is a pharmaceutically acceptable anion.

7. The method of any of claim 1-5 or 6, wherein the hemichannel blocker is administered orally in amount ranging from about 10 to 200 mg per dose.

8. The method of any of claim 1-5 or 6, wherein the hemichannel blocker is administered orally in amount ranging from about 80 to 320 mg per day.

9. The method of any of claim 1-5 or 6, wherein the hemichannel blocker is administered orally in an amount ranging from about 0.2 mg/kg to about 5 mg/kg per dose or per day.

10. The method of claim 4, wherein the circulating concentration of tonabersat in the subject ranges from about 10 micromolar to about 90 micromolar.

11. The method of claim 1, wherein said hemichannel inhibitor is administered by injection.

12. The method of claim 1, wherein said hemichannel inhibitor is administered orally.

13. The method of claim 1, wherein the hemichannel inhibitor is administered once per day or once per week.

14. The method of claim 1, wherein the hemichannel inhibitor is administered more than once per day.

15. The method of claim 1, wherein the hemichannel inhibitor induces or promotes closure of a hemichannel.

16. The method of claim 1, wherein the hemichannel inhibitor blocks, inhibits or decreases hemichannel opening.

17. The method of claim 1, wherein the hemichannel inhibitor triggers, induces or promotes cellular internalization of a hemichannel.

18. The method of claim 1, wherein the disease, disorder or condition is proliferative vitreoretinopathy.

19. The method of claim 1, wherein the subject is a human.

20. A method for inhibiting epithelial-mesenchymal transition activity in a subject, comprising administering a hemichannel inhibitor to said subject in an amount effective to inhibit epithelial-mesenchymal transition activity.

21. The method of claim 20, wherein the hemichannel inhibitor is a connexin 43 hemichannel inhibitor.

22. The method of claim 20, wherein the hemichannel inhibitor is a small molecule hemichannel inhibitor.

23. The method of claim 20, wherein the hemichannel inhibitor is N-[(3S,4S)-6-acetyl-3-hydroxy-2,2-dimethyl-3,4-dihydrochromen-4-yl]-3-chloro-4-fluorobenzamide (tonabersat).

24. The method of claim 20, wherein the hemichannel inhibitor is a compound is a compound of Formula (I).

25. The method of claim 20, wherein the hemichannel inhibitor is a compound is a compound of Formula (II).

26. The method of any of claim 20 or 23, wherein the subject has a fibrotic disorder.

27. The method of any of claim 20 or 23, wherein the subject has a disorder characterized in part by pathological or unwanted epithelial-mesenchymal transition activity.

28. The method of claim 27, wherein the disorder is a kidney disorder.

29. The method of claim 27, wherein the disorder is a pulmonary disorder.

30. The method of claim 27, wherein the disorder is a hepatic disorder.
